(19) United States
(12) Patent Application Publication
(10) Pub. No.: US 2021/0219589 A1
(43) Pub. Date: Jul. 22, 2021

(54) CANNABINOID EMULSION PRODUCT AND PROCESS FOR MAKING THE SAME

(71) Applicant: Aceso Wellness LLC, Denver, CO (US)

(72) Inventor: Joseph Faries Denniston, V, Wheat Ridge, CO (US)

(21) Appl. No.: 17/224,277

(22) Filed: Apr. 7, 2021

Related U.S. Application Data
(63) Continuation-in-part of application No. 16/656,313, filed on Oct. 17, 2019.
Application No. 16/656,313 is a continuation of application No. 15/462,495, filed on Mar. 17, 2017, now Pat. No. 10,542,770.
(60) Provisional application No. 62/310,079, filed on Mar. 18, 2016.

(51) Int. Cl. A23L 33/16; A61K 9/46; A61K 31/198; A61K 31/519; A61K 36/53; A61K 36/185; A61K 38/48; A23L 2/40; A23L 2/52; A23L 33/155; A23L 33/15; A23L 33/115; A61K 31/51; A61K 36/73; A61K 31/593; A61K 31/714; A61K 36/752; A61K 31/4188; A61K 9/20; A61K 36/9066; A61K 31/455; A61K 31/07; A61K 31/4415; A61K 31/525; A61K 31/191; A61K 31/7008; A23L 2/395; A61K 36/81

(52) U.S. Cl. CPC A23L 33/16; A23V 2200/31; A61K 31/198; A61K 31/519; A61K 36/53; A61K 36/185; A61K 38/4873; C12Y 304/22032; C12Y 304/22033; A23L 2/40; A23L 2/52; A23L 33/155; A23L 33/15; A23L 33/115; A61K 31/51; A61K 36/73; A61K 31/593; A61K 31/714; A61K 36/752; A61K 31/4188; A61K 9/2009; A61K 36/9066; A61K 31/455; A61K 31/07; A61K 31/4415; A61K 31/525; A61K 31/191; C12Y 304/22004; A61K 31/7008; A23L 2/395; A61K 36/81; A23V 2002/00; A61K 9/0007

(57) ABSTRACT

A dry consumable preparation and related methods are disclosed. The preparation has a plurality of excipient particles and at least one of a cannabinoid and a cannabinoid extract, the at least one of the cannabinoid or the cannabinoid extract containing one or more cannabinoids plated to the plurality of excipient particles. The preparation further comprises an effervescence agent, the effervescence agent comprising a carbonate salt, and at least one acid. The carbonate salt comprises sodium, potassium and/or calcium. The at least one acid comprises at least one of citric acid, tartaric acid, lactic, acetic, benzoic, ascorbic, oxalic, tannic, phosphoric, or malic acid. A ratio of the at least one of the carbonate salt to the at least one acid is at least 1:1, the ratio configured to create a chemical pH buffering system at a targeted pH range when the dry consumable preparation is added to a targeted amount of water.

### CLAIM OF PRIORITY

This application is a continuation in part of U.S. application Ser. No. 16/656,313, filed Oct. 17, 2019 and entitled “Cannabinoid Emulsion Product and Process for Making the Same,” which is a continuation of U.S. application Ser. No. 15/462,495 filed Mar. 17, 2017 and entitled “Cannabinoid Emulsion Product and Process for Making the Same.” U.S. application Ser. No. 15/462,495, now issued as U.S. Pat. No. 10,542,770, claims priority to U.S. Provisional Application No. 62/310,079, entitled “Cannabinoid Emulsion Product and Process for Making the Same” and filed Mar. 18, 2016. All applications are incorporated herein by reference in their entirety.

### BACKGROUND

Many dietary supplements, food ingredients, and foods are referred to as “nutraceuticals.” An item is often referred to as a nutraceutical when it is believed to provide both a “nutritious” and “healthy” effect (i.e., the item has an effect similar to a pharmaceutical) to the person ingesting the item. Any food item which is purported to provide an extra health benefit in addition to the basic nutritional value of the food may be referred to as a nutraceutical.

Current products containing cannabinoids include edibles containing cannabis oil, powders and liquid emulsions. One problem with products on the market is that the cannabinoids are not soluble in water. Some products include an array of emulsifiers or surfactants such as glycerin, coconut oil, polysorbates, or other emulsifiers. These emulsified cannabinoids are most commonly used in liquid phase preparations for ease of use and maintaining the integrity of the emulsion. The powder preparations of cannabinoids are commonly prepared from purified and crystallized cannabinoids. These crystallized cannabinoids are not soluble in water. Additionally, powder preparations of cannabinoids create unstable emulsions when mixed with water.

Another problem with cannabinoid products on the market is that a large majority of them are sustained release formulations following a combination of sigmoidal and quadratic pharmacokinetic curves. In some circumstances, the onset and offset of effects over time for a sustained release formulation following a combination of sigmoidal and quadratic pharmacokinetic curves may not be as intuitive for a consumer. Additionally, the duration between onset and offset of effects may be relatively long (e.g., 8-10 hours), which may be unsuitable for some consumers.

### SUMMARY

The following presents a summary relating to one or more aspects and/or embodiments disclosed herein. As such, the following summary should not be considered an overview relating to all contemplated aspects and/or embodiments, nor should the following summary be regarded to elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below. For the purposes of this disclosure, the terms “excipient” or “excipient particles”, and “bulking agent” may be used interchangeably throughout this application.

Some embodiments of the disclosure may be characterized as dry consumable preparation comprising: a plurality of excipient particles; at least one of a cannabinoid and a cannabinoid extract containing one or more cannabinoids, the at least one of the cannabinoid or the cannabinoid extract containing one or more cannabinoids plated to the plurality of excipient particles; an effervescence agent, the effervescence agent comprising a carbonate salt and at least one acid; wherein, the carbonate salt comprises at least one of sodium, potassium and calcium; the at least one acid comprises at least one of citric acid, tartaric acid, lactic, acetic, benzoic, ascorbic, oxalic, tannic, phosphoric, or malic acid; and a ratio of the carbonate salt to the at least one acid is at least 1:1, the ratio configured to create a chemical pH buffering system at a targeted pH range when the dry consumable preparation is added to a targeted amount of water.

Another embodiment of the disclosure may be characterized as a method of producing a cannabinoid preparation, the method comprising: providing to a fluid bed dryer a powdered preparation, wherein, the powdered preparation comprises a plurality of excipient particles and at least one of a cannabinoid and a cannabinoid extract containing one or more cannabinoids plated to the plurality of excipient particles, the plurality of excipient particles comprise, an oil adsorbent agent, and an outer surface having one or more pores and capillaries, the at least one of the cannabinoid or the cannabinoid extract plated to the plurality of excipient particles comprises at least one of the cannabinoid or the cannabinoid extract containing one or more cannabinoids comprise plated to one of the outer surface, pores, and capillaries; spraying an aqueous solution onto the outer surface; drying the particles; and encapsulating the particles with a layer of functional ingredients.

### BRIEF DESCRIPTION OF THE DRAWINGS

Table 1 is an ingredients list for a dry preparation for calming;

Table 2 is an ingredients list for a dry preparation for general wellness;

Table 3 is an ingredients list for a dry preparation for soothing;

Table 4 is an ingredients list for another dry preparation for general wellness;

Table 5 is an ingredients list for another dry preparation for calming;

Table 6 is an ingredients list for another dry preparation for soothing;

FIG. 1 is a flowchart of a method of making a dry consumable preparation;

FIG. 2 illustrates a comparative graph of immediate vs sustained release pharmacokinetics, in accordance with one or more implementations;

FIG. 3 illustrates a process flow for making a cannabinoid emulsion product in accordance with one or more implementations;

FIG. 4 illustrates a fluid bed dryer for encapsulating particles, according to an embodiment of the disclosure.

FIG. 5 illustrates a method of producing a cannabinoid preparation, according to an embodiment of the disclosure;

FIG. 6 illustrates a method of producing a cannabinoid preparation, according to an embodiment of the disclosure.

### DETAILED DESCRIPTION

Cannabidiol (CBD) is one of at least eighty-five non-psychoactive chemical cannabinoid compounds found in a cannabis plant. CBD consumption has been shown to provide potential health benefits. For example, CBD may elicit antidepressant, anxiolytic, and neuroprotective effects on its users. Additional beneficial effects may also occur. Given the beneficial effects from CBD, it is contemplated to create one or more nutraceutical items that include CBD and other ingredients. One or more of the other ingredients may include other non-psychoactive cannabinoids such as, but not limited to, cannabigerol (CBG), cannabinol (CBN) and/or cannabichromene (CBC).

The insolubility of cannabinoids in water and the requirement for emulsifiers to maintain the cannabinoids in a water cannabinoid emulsion have prevented the creation of powder preparations of cannabinoids that are easily mixed and also create stable emulsions in water, thus presenting a long-felt and unmet need for a product that provides these benefits. The Applicant has created a dry preparation of cannabinoids and other nutrients. The formulation of the dry preparation may allow for easy mixing and create a stable emulsion in water. Some embodiments of the dry preparation are prepared so as to result in a liquid having an effervescence. Further, upon mixing with a liquid, some formulations of the Applicant's dry preparation may create a stable emulsion that resists the changes of pH after effervescence, and changes in other chemical characteristics of the solution that occur during effervescence.

The dry preparation may include a powder, a dissolvable capsule, and/or a tablet. The powder may be a loose powder. The capsule may include a gelatin coating. The capsule may encapsulate a powder. The tablet may be a compacted or pressed tablet. In some embodiments, the liquid may include water only. In some embodiments, the liquid may include tap water, purified water, mineral water, spring water, and/or any drinking water. In some embodiments, the liquid may include a liquid suitable for drinking, such as, but not limited to, water, milk, soda, and/or or fruit juice.

In some cases, the beverage powder may comprise additional functional ingredients (e.g., sweeteners) as compared to the tablets and/or capsules, since the latter may not have enough mass to support the inclusion of effective amounts of functional ingredients, such as effervescent agents, vitamins, acidulants, sweeteners, flavors, and/or botanical extracts to name a few non-limiting examples. Alternatively, the capsules and/or tablet may be of a larger size than a typical oral tablet (e.g., less than 1 gram) so additional functional ingredients may be included in the tablet or capsule. In some cases, the typical bulking agent:active cannabinoid ingredient ratio in a tablet may be anywhere from about 50:1 to about 200:1. In contrast, the typical bulking agent:active cannabinoid ingredient ratio in the present disclosure may be anywhere from about 400:1 to about 600:1. One non-limiting example of the additional bulking agent may comprise sugar for providing sweetness to the beverage. In some cases, typical oral tablets or capsules, including dissolvable capsules/tablets, in the prior art may not include beverage ingredients, also referred to herein as factors, such as sweeteners, acidulant, color, and/or flavor. For instance, a single serving of a tablet or capsule may contain up to ten times less mass than a single serving of a beverage powder, which may make the inclusion of one or more critical beverage factors unfeasible. Additionally, or alternatively, since the binder:active cannabinoid ingredient ratio is lower in a tablet or capsule as compared to a beverage powder, one or more critical beverage factors may not be included. It is contemplated that when used herein, the term “about”, and other similar terms, refers to a range of +/−1% of any stated value or amount, more preferably refers to a range of 0.1% of any stated value or amount, and most preferably refers to a range of +/1.01% of any stated value or amount.

In some embodiments, a dry consumable preparation 1000, 2000, 3000, 4000, 5000, 6000 is provided. In some cases, an excipient may refer to a substance included in a powder, capsule, and/or a tablet that is included primarily to aid in the manufacturing process, protect, support, and/or enhance stability rather than to provide therapeutic or potential benefits. From a therapeutic or health benefits standpoint, the excipient may serve as an inactive ingredient and may be used to bulk, bind, coat, impart color and/or flavor to the dry preparation to name a few non-limiting examples. In some examples, the dry consumable preparation may comprise a plurality of excipient particles, where at least a portion of the excipient particles may serve as a bulking agent, at least one of a cannabinoid or a cannabinoid extract having one or more cannabinoids, and an effervescence agent. In some cases, the at least one of the cannabinoid or the cannabinoid extract may be plated to the plurality of excipient particles (or the bulking agent). In some cases, the excipient may be an adsorbent excipient and the cannabinoid may be plated onto the adsorbent excipient. In addition to the bulking agent, some non-limiting examples of excipients may include binders, coatings, colors, flavors, sorbents, and sweeteners (e.g., sugar, sucralose, acesulfame potassium, etc.). In some embodiments, the ratio of the bulking agent to the active cannabinoid ingredients, for instance, on a dry-weight basis, may be anywhere from between 400:1 and 600:1. It should be noted that different ratios of bulking agents to active cannabinoid ingredients are contemplated in different embodiments. In some cases, sugar may also be utilized as an additional bulking agent, which may serve to provide sweetness to the beverage.

Those skilled in the art will recognize that plating is the process of turning a liquid or oil into a dry powder; specifically, the oil or liquid is extended onto a dry edible carrier, and the resulting mixture behaves substantially as a powder would. In some embodiments, the surface of an individual particle of excipient can be coated with cannabinoid oil. The excipient may possess adsorbent properties to retain the oil without swelling greater than 50% in volume. In some examples, a portion of the oil may be coated onto the surface of the excipient particle while another portion may be adsorbed into the inner pores and capillaries within the three-dimensional inner structure of the excipient particle. In some aspects, coating and adsorption of the oil within the inner pores and capillaries of the porous excipient may serve to provide protection from ultraviolet (UV) radiation and/or oxygen. The ratio of adsorbed oil to total oil may vary depending on the excipient's structural porosity and/or oil adsorbent capabilities, to name two non-limiting factors.

In some examples, an additional encapsulant is coupled to an outer surface of one or more of the at least one of a cannabinoid or a cannabinoid extract containing one or more cannabinoids and the plurality of excipient particles. The plurality of excipient particles may comprise at least one of a carbohydrate sweetener, a water-soluble powdered flavor, and a water-soluble powdered color. Some non-limiting examples of the carbohydrate sweetener may comprise at least one of sucrose, fructose, glucose, corn syrup, corn syrup solids, lactose, honey, agave nectar, allulose, maltodextrin, tapioca syrup, digestion resistant maltodextrin, trehalose, galactose, and starch. Further, the encapsulant may comprise a water-soluble starch. Additionally, or alternatively, the encapsulant may comprise an oxidative barrier, a flow aid, a bulking agent, a dissolution agent, and a carrier of active ingredients, wherein, the active ingredients comprise one or more of a dietary mineral content (e.g., Iron supplement, Zinc supplement, etc.) and an effervescence agent.

The effervescence agent may comprise sodium bicarbonate, potassium bicarbonate, and at least one acid. Additionally, or alternatively, the effervescence agent may comprise a carbonate salt and at least one acid. Some examples of the carbonate salt may comprise one or more of sodium, potassium and calcium. For instance, sodium carbonate, potassium carbonate, and/or calcium carbonate may be some non-limiting examples of the carbonate salts. In some examples, the acid may include citric acid, tartaric acid, lactic acid, acetic acid, benzoic acid, ascorbic acid, oxalic acid, tannic acid, phosphoric acid, and/or malic acid. In one example, the ratio of sodium bicarbonate to potassium bicarbonate to acid in the effervescence agent may be selected such that a chemical pH buffering system at a targeted pH range may be created when the dry consumable preparation is added to a targeted amount of water. In some cases, the bulking agent may include a sugar, such as fructose, although other types of bulking agents are contemplated in different embodiments.

In some instances, a ratio of the carbonate salt to the at least one acid may be at least 1:1, although other ratios of carbonate salt to acid are also contemplated. In some cases, the ratio of carbonate salt to acid may be selected to create a chemical pH buffering system at a targeted pH range when the dry consumable preparation is added to a targeted amount of water. One ratio of the water to the effervescence agent may be as high as 50:1 or as low as 350:1, and more preferably from 75:1 to 200:1 and most preferably around 100:1. Such a ratio may comprise the ratio of the weight of to six ounces of water to the weight of the dry consumable preparation or effervescent ingredients. In one example, the targeted pH range may be anywhere between 2 and 6 (i.e., acidic). For the purposes of this disclosure, the ratio of the carbonate salt to the at least one acid may refer to a dry ratio of the individual components. In one example, the tartaric acid may comprise 2.7% of the total dry weight of the powdered preparation. In other words, the ratio of tartaric acid to the whole formulation (i.e., dry consumable preparation) may be about 0.027:1. In some embodiments, a nutrient, such as bromelain may be used in combination with the tartaric acid. In one example, the amount of bromelain used may be about 0.3%. In this case, the ratio of tartaric acid to the bromelain may be about 9:1.

Three formulations are described herein generally, with ingredients for General Wellness 2000, 4000, Calming 1000, 5000, and Soothe 3000, 6000, with each having variations within. Each of these are formulated for the specific purpose of supporting general wellness and energy, supporting a calm and improved mood, and supporting joint health and combating inflammation, respectively.

In some embodiments, the effervescence may enhance uptake of cannabinoids and buffering stomach acids, thereby protecting acid label cannabinoids.

Some examples described herein include CBD-based effervescent dry preparations and other products. Some dry preparations may provide various nutraceutical benefits to the user through the incorporation of one or more ingredients in addition to the CBD. For instance, the dry consumable preparation may comprise a nutraceutical composition, the nutraceutical composition having an amount of Quillaja selected to impart a stabilizing and emulsifying effect when the preparation is mixed with a targeted amount of water. In one example, the amount of Quillaja by dry weight may range from about 0.03% to about 0.06%, although other dry-weight ranges are contemplated in different embodiments.

One such nutraceutical item may include an effervescent powder. The ingredients in an effervescent powder may include an acid and a base mixture. Upon mixing with water or another liquid, the acid and base mixture may interact, thereby producing carbon dioxide gas, which may accumulate and form bubbles. Those skilled in the art will recognize that the effervescent powder should be selected so as to provide an aqueous chemical environment that does not break the emulsion.

In some embodiments, an acid in the dry preparation may include citric acid. In some embodiments, the base in the dry preparation may include one or more bicarbonates such as, but not limited to, calcium bicarbonate, sodium bicarbonate, and/or potassium bicarbonate. In some embodiments, 1%, and up to 15%, or about 7%, of the effervescent powder may include the dried emulsion ingredients. Further ingredients may include vitamins, sugar and essential oil-based ingredients such as, but not limited to, terpenes. Terpenes may provide a nutraceutical synergy with CBD. For example, Beta-caryophyllene may include a terpene that may be included in the dry preparation. Beta-caryophyllene may be derived from a cinnamon essential oil. Additional ingredients adapted to stabilize the emulsion after mixing may also be included in the dry preparation. Additional ingredients may also be included in the dry preparation. For example, maltodextrin may be utilized as an ingredient. In some examples, the dry preparation may comprise about 10% maltodextrin by weight.

As previously described, upon mixing the powder or tablet with a liquid such as, but not limited to, water, a bubbly and fizzy consumable mixture may be created. To realize the nutraceutical benefits associated with the dry preparation, a user may ingest the mixture immediately after mixing, during effervescence, or wait until after the effervescence has ceased, or wait about two to three minutes after the effervescence has ceased.

It may be preferable to consume during effervescence, which may impart advantages in palatability and delivery and absorption of the cannabinoid mixture and other nutrients within the formula.

In some embodiments, a consumable product having a liquid oil/water emulsion for use as a spray is provided. That is, a liquid emulsion having at least some of the features previously described as they relate to the dry preparation and emulsion may be provided, for use as a spray. In some embodiments, the spray may include an aqueous solution having a translucent or non-milky appearance. The spray may be used for general wellness, a calming effect, pain relief, and/or sleep benefits.

There may be a synergistic general wellness relationship between various terpenes and cannabinoids, and, in some embodiments, the cyclic terpene limonene may be used to provide one of these benefits, or any other benefit.

Some embodiments provide a water-based frozen consumable such, as, but not limited to, an ice pop. In some embodiments, the water-based frozen consumable may have an emulsion. In some embodiments, the water-based frozen consumable may have an effervescence upon melting in one's mouth. The water-based frozen consumable may have substantially all the ingredients described herein as they relate to an effervescent powder or tablet or emulsion. In addition to including one or more of the ingredients listed above, such as cannabidiol, which reduces muscle inflammation, other ingredients in the water-based frozen consumable may include flavored salts. The water-based frozen consumable may provide benefits to endurance athletes, cancer patients and/or other patients with a debilitating condition.

Those skilled in the art will recognize that the water-based frozen consumable may include a frozen emulsion where the emulsion is adapted to survive the liquid-to-solid phase transition. For example, oil may be added to an emulsifier at a high temperature, but without breaking the emulsion. An emulsifier may include a natural emulsifier such as, but not limited to, natural Quillaja tree extract, and/or any other emulsifier with a high HLB ratio. Quillaja may also provide a stabilizing effect on the resulting effervescent drink. In some embodiments, the amount of Quillaja may be directly related to the amount of cannabis oil being used, which may in turn be based on the cannabinoid potency of the oil. In one example, the amount of Quillaja may range from about 0.03% to about 0.06% on a dry weight basis.

The HLB ratio is the ratio of Hydrophile-Lipophile Balance, which is an empirical expression for the relationship between the hydrophilic (“water-loving”) and hydrophobic (“water-hating”) groups of a surfactant. The higher the HLB ratio, the more water-soluble the surfactant or emulsifier.

In some embodiments, the water-based frozen consumable may include an emulsion created by using a high-shear mixer. Upon freezing, the oil in the mixture may be encapsulated by ice crystals, preventing oil from creating a layer on the ice pop packaging.

As previously described, some embodiments of the dry preparation may include a powder. The powder may include a fine, free-flowing effervescent, nutritional beverage powder infused with CBD. Such a powder may be combined with a person's desired liquid to create a beverage. One type of liquid may include water. Various ingredients may be needed to create such a powder.

For example, the powder may include one or more of: Sodium citrate, Magna Sweet, L-theanine, Passion flower flavor powder, Grapefruit essential oil, Lavender essential oil, Fructose, Sodium Bicarbonate, Calcium Carbonate, Potassium Carbonate, Citric Acid, Vitamin C, Coenzyme Q10, CBD oil, Quillaja, Cayenne extract, Lemon oil, Orange oil, Malic acid, Honey powder, Vitamin A, Vitamin D3, Vitamin B6, Vitamin B3, Vitamin B2, Vitamin B1 ,Vitamin B12, Zinc gluconate, Biotin, cherry flavor powder or other flavor powder, glucosamine sulfate, cinnamon essential oil, turmeric extract, and bromelain (600 GDU).

In some embodiments, the dry preparation 1000, 2000, 3000, 4000, 5000, 6000 may include more than 0.1% by weight of CBD oil. In some embodiments, the dry preparation may include more than 0.15% by weight CBD oil. In some embodiments, the dry preparation may have up to 5% by weight CBD oil. In some embodiments, the dry preparation may have between 0.1% and 5% by weight CBD oil, or between 0.1% and 2% by weight CBD oil, or between 2% and 5% by weight CBD oil. In some embodiments, the dry preparation may have about 0.155% by weight CBD oil, or about 0.15% by weight CBD oil, or between about 0.05% and about 0.3% by weight CBD oil.

Those skilled in the art will recognize that the presence of Tetrahydrocannabinol (THC) above 0.3% in the final product is prohibited by federal law. Therefore, if THC is present in the CBD oil, a dilution factor must be sufficient to dilute the THC to a level below 0.3%. Should federal law change, those skilled in the art may recognize that particular ratios of THC to CBD may be particularly beneficial; however, embodiments described herein may be specifically designed to deliver CBD, and not any relevant amounts of THC.

The preparation 1000, 2000, 3000, 4000, 5000, 6000 may have a nutraceutical composition, the nutraceutical composition having an amount of Quillaja selected to impart a stabilizing and emulsifying effect when the preparation is mixed with a targeted amount of water. The preparation may also have one or more herbal extracts, and one or more nutrient vitamins or minerals, and may be formulated to form a stable, effervescent emulsion when mixed with a targeted amount of water.

Dry Preparation for Calming

Turning now to Table 1, examples of a dry preparation for calming 1000 are described. A dry preparation having the ingredients illustrated in Table 1 may produce a calming effect upon ingesting. Three embodiments of the dry preparation for calming are illustrated. Embodiment 3 is illustrated in both weight in grams and weight by ratio.

The dry preparation for calming 1000 may include between about 0.03% and about 0.05% by weight sodium citrate or about 0.0493% by weight sodium citrate. The dry preparation for calming may include between about 0.2% and about 0.5% by weight vitamin C, or about 0.3% by weight vitamin C. The dry preparation for calming 1000 may include between about 0.2% and about 0.5% by weight coenzyme Q10, or about 0.332% by weight coenzyme Q10. The dry preparation for calming 1000 may include between about 0.05% and about 0.2% by weight magna sweet, or about 0.113% by weight magna sweet. The dry preparation for calming 1000 may include between about 0.2% and about 0.5% by weight L Theanine, or about 0.331% by weight L Theanine. The dry preparation for calming 1000 may include between about 0.1% and about 0.3% by weight passionflower flavor powder or about 0.2% weight of passionflower flavor powder. The dry preparation for calming 1000 may include between about 0.05% and about 0.25% by weight CBD Oil (64.5%), or about 0.155% by weight CBD Oil (64.5% CBD). The dry preparation for calming 1000 may include between about 70% and about 80% by weight fructose, or about 76.036% by weight fructose. The dry preparation for calming 1000 may include between about 0.3% and about 0.5% by weight grapefruit oil, or about 0.429% by weight grapefruit oil. The dry preparation for calming 1000 may include between about 0.02% and about 0.04% by weight lavender oil, or about 0.029% by weight lavender oil. The dry preparation for calming 1000 may include between about 17% and about 19% by weight citric acid or about 17.699% by weight citric acid. The dry preparation for calming 1000 may include between about 0.7% and about 1.0% by weight sodium bicarbonate, or about 0.885% by weight sodium bicarbonate. The dry preparation for calming 1000 may include between about 0.8% and about 1.3% by weight calcium carbonate, or about 1.063% by weight calcium carbonate. The dry preparation for calming 1000 may have between about 2.0% and about 2.8% by weight potassium bicarbonate, or about 2.302% by weight potassium bicarbonate. The dry preparation for calming 1000 may have between about 0.05% and about 0.15% by weight Quillaja, or about 0.077% by weight Quillaja.

The dry preparation for calming 1000 may have a ratio of grapefruit oil to lavender oil of about 15:1. The dry preparation for calming 1000 may have a ratio of calcium carbonate to sodium bicarbonate of between about 1:1 and 2:1, or about 1.2:1. The dry preparation for calming 1000 may have a ratio of potassium bicarbonate to sodium bicarbonate of between about 2:1 and 3:1, or about 2.6:1. The dry preparation for calming 1000 may have a ratio of Quillaja to CBD oil of between about 1:1 and 1:4, or about 1:2.

Turning now to Table 2, examples of a dry preparation for general wellness 2000 are described. A dry preparation having the ingredients illustrated in Table 2 may provide wellness benefits upon ingesting on a short-term or long-term basis. Three embodiments of the dry preparation for wellness 2000 are illustrated. Embodiment 3 is illustrated in both weight in grams and weight by ratio.

The dry preparation for wellness 2000 may have between about 0.001% and about 0.003% by weight Vitamin A, or about 0.0027% by weight Vitamin A. The dry preparation for wellness 2000 may have between about 0.0001% and about 0.0003% by weight Vitamin D3, or about 0.0002% by weight Vitamin D3. The dry preparation for wellness 2000 may have between about 0.004% and about 0.008% by weight Vitamin B6, or about 0.006% by weight Vitamin B6. The dry preparation for wellness 2000 may have between about 0.03% and about 0.07% by weight Vitamin B3, or about 0.048% by weight Vitamin B3. The dry preparation for wellness 2000 may have between about 0.005% and about 0.03% by weight Vitamin B2, or about 0.0173% Vitamin B2. The dry preparation for wellness 2000 may have between about 0.005% and about 0.03% by weight Vitamin B1, or about 0.0173% by weight Vitamin B1. The dry preparation for wellness 2000 may have between about 0.0004% and about 0.0008% by weight Vitamin B12, or about 0.0006% by weight Vitamin B12. The dry preparation for wellness may have between about 0.1% and about 0.5% by weight Vitamin C, or about 0.30% by weight Vitamin C. The dry preparation for wellness 2000 may have between about 0.01% and about 0.04% by weight zinc gluconate, or about 0.024% by weight zinc gluconate. The dry preparation for wellness 2000 may have between about 0.005% and about 0.003% by weight Biotin, or about 0.0013% by weight Biotin. The dry preparation for wellness 2000 may have between about 0.3% and about 0.7% by weight malic acid, or about 0.57% by weight malic acid. The dry preparation for wellness 2000 may have between about 0.05% and about 0.2% by weight sodium citrate, or about 0.11% by weight sodium citrate. The dry preparation may have between about 0.04% and about 0.09% by weight honey powder, or about 0.07% by weight honey powder. The dry preparation for wellness 2000 may have between about 0.05% and about 0.3% by weight magna sweet, or about 0.14% by weight magna sweet. The dry preparation for wellness 2000 may have between about 0.1% and about 0.3% by weight green tea flavor powder or other flavor, or about 0.20% by weight green tea flavor powder or other flavor. The dry preparation for wellness 2000 may have between about 0.05% and about 0.5% by weight CBD oil, or about 0.15% by weight CBD oil. The dry preparation for wellness 2000 may have between about 0.005% and about 0.2% by weight Quillaja, or about 0.08% by weight Quillaja. The dry preparation for wellness 2000 may have between about 0.2% and about 0.6% by weight lemon oil, or about 0.47% by weight lemon oil. The dry preparation for wellness 2000 may have between about 0.2% and about 0.5% by weight orange oil, or about 0.36% by weight orange oil. The dry preparation for wellness 2000 may have between about 0.05% and about 0.3% by weight cayenne extract, or about 0.16% by weight cayenne extract. The dry preparation for wellness 2000 may have between about 68% and about 79% by weight fructose, or about 73.62% by weight fructose. The dry preparation for wellness 2000 may have between about 1% and about 2% by weight sodium bicarbonate, or about 1.57% by weight sodium bicarbonate. The dry preparation for wellness 2000 may have between about 0.5% and about 1.5% by weight calcium carbonate, or about 1.08% by weight calcium carbonate. The dry preparation for wellness 2000 may have between about 2.5% and about 3.5% by weight potassium bicarbonate, or about 3.13% by weight potassium bicarbonate. The dry preparation for wellness 2000 may have between about 16.5% and about 18.5% by weight citric acid, or about 17.86% by weight citric acid.

Turning now to Table 3, examples of a dry preparation for soothing 3000 are described. A dry preparation having the ingredients illustrated in Table 3 may provide benefits upon ingesting on a short-term or long-term basis. The benefits may include but are not limited to reduction in inflammation, strong cartilage and healthy joints, and an increase in antioxidant activity, preventing cellular damage and stress. Three embodiments of the dry preparation for soothing 3000 are illustrated. Embodiment 3 is illustrated in both weight in grams and weight by ratio.

The dry preparation for soothing 3000 may have between about 0.5% and about 1.5% by weight sodium citrate, or about 0.12% by weight sodium citrate. The dry preparation for soothing 3000 may have between about 0.2% and about 0.4% by weight Vitamin C, or about 0.3% by weight Vitamin C. The dry preparation for soothing 3000 may have between about 0.1% and about 0.3% by weight cherry flavor or other flavor powder, or about 0.2% by weight cherry flavor or other flavor powder. The dry preparation for soothing 3000 may have between about 0.5% and about 0.2% by weight CBD oil, or about 0.15% by weight CBD oil. The dry preparation for soothing 3000 may have between about 0.9% and about 2.0% glucosamine sulfate, or about 1.33% by weight glucosamine sulfate. The dry preparation for soothing 3000 may have between about 0.0001% and about 0.0005% Vitamin D, or about 0.0003% by weight Vitamin D. The dry preparation for soothing 3000 may have between about 0.05% and about 0.2% by weight bromelain, or about 0.13% by weight bromelain. The dry preparation for soothing 3000 may have between about 0.05% and about 0.09% by weight Quillaja, or about 0.08% by weight Quillaja. The dry preparation for soothing 3000 may have between about 0.07% and about 0.15% by weight magna sweet, or about 0.09% by weight magna sweet. The dry preparation for soothing 3000 may have between about 75% and about 80% by weight fructose G, or about 78.37% by weight fructose G. The dry preparation for soothing 3000 may have between about 0.1% and about 0.4% by weight cinnamon oil, or about 0.28% by weight cinnamon oil. The dry preparation for soothing 3000 may have between about 0.5% and about 1.2% by weight turmeric extract, or about 0.77% by weight turmeric extract. The dry preparation for soothing 3000 may have between about 13% and about 16% by weight citric acid, or about 14.27% by weight citric acid. The dry preparation for soothing 3000 may have between about 0.8% and about 1.5% by weight sodium bicarbonate, or about 1.19% by weight sodium bicarbonate. The dry preparation for soothing 3000 may have between about 0.5% and about 1.3% by weight calcium carbonate, or about 0.86% by weight calcium carbonate. The dry preparation for soothing 3000 may have between about 1.4% and about 2.5% by weight potassium bicarbonate, or about 1.85% by weight potassium bicarbonate.

Turning now to Table 4, examples of another dry preparation for wellness 4000 are described. A dry preparation having the ingredients illustrated in Table 4 may provide benefits upon ingesting on a short-term or long-term basis. Three embodiments of the dry preparation for wellness 4000 are illustrated. Embodiment 3 is illustrated in both weight in milligrams and weight by ratio.

The dry preparation for wellness 4000 may have between about 0.0005% and about 0.002% by weight Biotin or Vitamin B7, or about 0.001% by weight Biotin or Vitamin B7. The dry preparation for wellness 4000 may have between about 0.002% and about 0.004% by weight Vitamin A or Palmitate, or about 0.003% by weight Vitamin A or Palmitate. The dry preparation for wellness 4000 may have between about 0.015% and about 0.03% by weight Vitamin B1 or Thiamine HCL, or about 0.02% by weight Vitamin B1 or Thiamine HCL. The dry preparation for wellness 4000 may have between about 0.015% and about 0.03% by weight Vitamin B2 or Riboflavin, or about 0.03% by weight Vitamin B2 or Riboflavin. The dry preparation for wellness 4000 may have between about 0.04% and about 0.06% by weight Vitamin B3 or Niacin USP or about 0.05% by weight Vitamin B3 or Niacin USP. The dry preparation for wellness 4000 may have between about 0.05% and about 0.07% by weight Vitamin B6 or Pyridoxine HCL, or about 0.06% by weight Vitamin B6 or Pyridoxine HCL. The dry preparation for wellness 4000 may have between about 0.0005% and about 0.003% by weight Vitamin B12 or Cyanocobalamin, or about 0.001% Vitamin B12 or Cyanocobalamin. The dry preparation for wellness 4000 may have between about 2% and about 20% by weight Vitamin C or ascorbic acid, or about 9.94% by weight Vitamin C or ascorbic acid. The dry preparation for wellness 4000 may have between about 0.00005% and about 0.0002% by weight Vitamin D3 or cholecalciferol, or about 0.0001% by weight Vitamin D3 or cholecalciferol. The dry preparation for wellness 4000 may have between about 0.015% and about 0.03% by weight zinc gluconate, or about 0.02% by weight zinc gluconate. The dry preparation for wellness 4000 may have between about 2% and about 15% by weight sodium bicarbonate, or about 5.3% by weight sodium bicarbonate. The dry preparation for wellness 4000 may have between about 1% and about 8% by weight potassium bicarbonate, or about 1.33% by weight potassium bicarbonate. The dry preparation for wellness 4000 may have between about 5% and 10% by weight citric acid, or about 7.95% by weight citric acid. The dry preparation for wellness 4000 may have between about 4% and about 10% by weight tartaric acid, or about 5.3% by weight tartaric acid. The dry preparation for wellness 4000 may have between about 0.15% and about 0.35% by weight CBD oil (50%, 10 mg) C16-60, or about 0.25% by weight CBD Oil. The dry preparation for wellness 4000 may have between about 0.5% and about 1.5% by weight cayenne extract (80% alcohol), or about 0.8% by weight cayenne extract. The dry preparation for wellness 4000 may have between about 0.005% and about 0.1% by weight lemon oil, or about 0.07% by weight lemon oil. The dry preparation for wellness 4000 may have between about 0.1% and about 0.4% Quillaja, or about 0.25% by weight Quillaja. The dry preparation for wellness 4000 may have between about 0.5% and about 5% by weight MET5207 or honey flavor or other flavor, or about 1.33% by weight MET5207 or honey flavor or other flavor. The dry preparation for wellness 4000 may have between about 0.3% and about 1% by weight MET3636 or green tea flavor, or about 0.66% by weight MET3636 or green tea flavor. The dry preparation for wellness 4000 may have between about 3% and about 6% by weight MET0340 or lemon flavor or other flavor, or about 4.97% by weight MET0340 or lemon flavor or other flavor. The dry preparation for wellness 4000 may have between about 0.1% and about 0.5% by weight MET0983 or yellow color, or about 0.2% by weight MET0983 or yellow color. The dry preparation for wellness may have between about 30% and about 35% by weight fructose, or about 30.74% by weight fructose. The dry preparation for wellness may have between about 30% and about 35% by weight sucrose, or about 30.74% by weight sucrose.

The dry preparation for wellness 4000 may have a ratio of sodium bicarbonate to potassium bicarbonate of between about 2:1 and about 5:1. The dry preparation for wellness 4000 may have a ratio of sodium bicarbonate to potassium bicarbonate of about 4:1. The dry preparation for wellness 4000 may have a ratio of Quillaja to CBD oil of between about 1:1 and about 1:4. The dry preparation for wellness 4000 may have a ratio of Quillaja to CBD oil of about 1:2. The dry preparation for wellness 4000 may have a ratio of fructose to sucrose of between about 1:0.75 and about 1:1. The dry preparation for wellness 4000 may have a ratio of fructose to sucrose of about 1:1.

Turning now to Table 5, examples of another dry preparation for calming 5000 are described. A dry preparation having the ingredients illustrated in Table 5 may provide benefits upon ingesting on a short-term or long-term basis. Three embodiments of the dry preparation for calming 5000 are illustrated. Embodiment 3 is illustrated in both weight in milligrams and weight by ratio.

The dry preparation for calming 5000 may have between about 7% and about 9% by weight Vitamin C or ascorbic acid, or about 7.98% by weight Vitamin C or ascorbic acid. The dry preparation for calming 5000 may have between about 0.1% and about 0.5% by weight L-Theanine, or about 0.33% by weight L-Theanine. The dry preparation for calming 5000 may have between about 0.005% and about 0.02% by weight folic acid, or about 0.01% by weight folic acid. The dry preparation for calming 5000 may have between about 3% and about 7% by weight sodium bicarbonate, or about 5.32% by weight sodium bicarbonate. The dry preparation for calming 5000 may have between about 0.9% and about 2% by weight potassium bicarbonate, or about 1.33% by weight potassium bicarbonate. The dry preparation for calming 5000 may have between about 6% and about 9% citric acid, or about 7.98% by weight citric acid. The dry preparation for calming 5000 may have between about 4% and about 7% by weight tartaric acid, or about 5.32% by weight tartaric acid. The dry preparation for calming 5000 may have between about 0.15% and about 0.4% by weight CBD oil (50%, 10mg) C16-60, or about 0.27% by weight CBD oil. The dry preparation for calming 5000 may have between about 0.005% and about 0.09% by weight lavender oil, or about 0.03% by weight lavender oil. The dry preparation for calming 5000 may have between about 0.05% and about 0.3% by weight Quillaja, or about 0.15% by weight Quillaja. The dry preparation for calming 5000 may have between about 0.05% and about 0.3% by weight passionflower extract, or about 0.11% by weight passionflower extract. The dry preparation for calming 5000 may have between about 4% and about 10% by weight MET0839 or grapefruit flavor or other flavor, or about 7.32% by weight MET0839 or grapefruit flavor or other flavor. The dry preparation for calming 5000 may have between about 0.005% and about 0.1% by weight MET3805 or beet powder, or about 0.01% by weight MET3805 or beet powder. The dry preparation for calming 5000 may have between about 30% and about 35% by weight fructose, or about 31.93% by weight fructose. The dry preparation for calming 5000 may have between about 30% and about 35% by weight sucrose, or about 31.93% by weight sucrose.

The dry preparation for calming 5000 may have a ratio of sodium bicarbonate to potassium bicarbonate of between about 2:1 and about 5:1. The dry preparation for calming 5000 may have a ratio of sodium bicarbonate to potassium bicarbonate of about 4:1. The dry preparation for calming 5000 may have a ratio of Quillaja to CBD oil of between about 1:1 and about 1:4. The dry preparation for calming 5000 may have a ratio of Quillaja to CBD oil of about 1:2. The dry preparation for calming 5000 may have a ratio of fructose to sucrose of between about 1:0.75 and about 1:1.25. The dry preparation for calming 5000 may have a ratio of fructose to sucrose of about 1:1.

Turning now to Table 6, examples of another dry preparation for soothing 6000 are described. A dry preparation having the ingredients illustrated in Table 6 may provide benefits upon ingesting on a short-term or long-term basis. Three embodiments of the dry preparation for soothing 6000 are illustrated. Embodiment 3 is illustrated in both weight in milligrams and weight by ratio.

The dry preparation for soothing 6000 may have between about 5% and about 7% by weight Vitamin C, or about 6.66% by weight Vitamin C. The dry preparation for soothing 6000 may have between about 1% and about 3% by weight glucosamine sulfate, or about 1.33% by weight glucosamine sulfate. The dry preparation for soothing 6000 may have between about 0.0001% and about 0.0007% by weight Vitamin D, or about 0.0003% by weight Vitamin D. The dry preparation for soothing 6000 may have between about 0.05% and about 0.2% by weight bromelain, or about 0.13% by weight bromelain. The dry preparation for soothing 6000 may have between about 4% and about 6% by weight sodium bicarbonate, or about 5.33% by weight sodium bicarbonate. The dry preparation for soothing 6000 may have between about 0.9% and about 2% by weight potassium bicarbonate, or about 1.33% by weight potassium bicarbonate.

The dry preparation for soothing 6000 may have between about 6% and about 10% by weight citric acid, or about 8% by weight citric acid. The dry preparation for soothing 6000 may have between about 1% and about 4% by weight tartaric acid, or about 2.67% by weight tartaric acid. The dry preparation for soothing 6000 may have between about 2.5% and about 4.5% by weight malic acid, or about 3.6% by weight malic acid. The dry preparation for soothing 6000 may have between about 0.1% and about 0.35% by weight CBD oil (50%, 10mg) C16-60, or about 0.27% by weight CBD oil. The dry preparation for soothing 6000 may have between about 0.1% and about 0.3% by weight turmeric extract, or about 0.2% by weight turmeric extract. The dry preparation for soothing 6000 may have between about 0.05% and about 0.15% by weight cinnamon oil, or about 0.09% by weight cinnamon oil. The dry preparation for soothing 6000 may have between about 0.1% and about 0.5% by weight Quillaja, or about 0.28% by weight Quillaja. The dry preparation for soothing 6000 may have between about 6% and about 10% MET6376 or cherry flavor or other flavor, or about 7.33% by weight MET6376 or cherry flavor or other flavor. The dry preparation for soothing 6000 may have between about 0.2% and about 0.9% by weight MET7987 or red color or other color, or about 0.4% by weight MET7987 or red color or other color. The dry preparation for soothing 6000 may have between about 30% and about 35% by weight fructose, or about 31.19% by weight fructose. The dry preparation for soothing 6000 may have between about 30% and about 35% by weight sucrose, or about 31.19% by weight sucrose.

The dry preparation for soothing 6000 may have a ratio of sodium bicarbonate to potassium bicarbonate of between about 2:1 and about 5:1. The dry preparation for soothing 6000 may have a ratio of sodium bicarbonate to potassium bicarbonate of about 4:1. The dry preparation for soothing 6000 may have a ratio of Quillaja to CBD oil of between about 1:1 and about 1:4. The dry preparation for soothing 6000 may have a ratio of Quillaja to CBD oil of about 1:2. The dry preparation for soothing 6000 may have a ratio of fructose to sucrose of between about 1:0.75 and about 1:1.25. The dry preparation for soothing 6000 may have a ratio of fructose to sucrose of about 1:1.

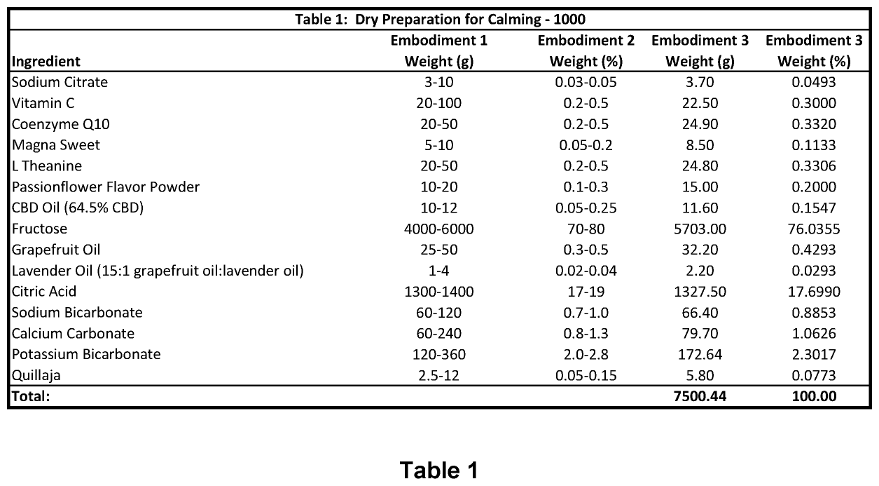

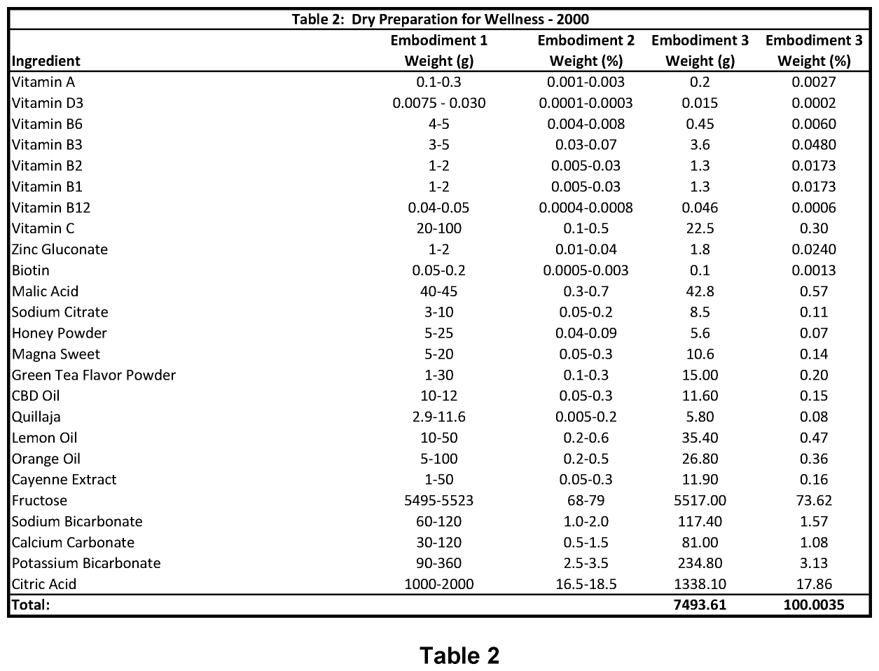

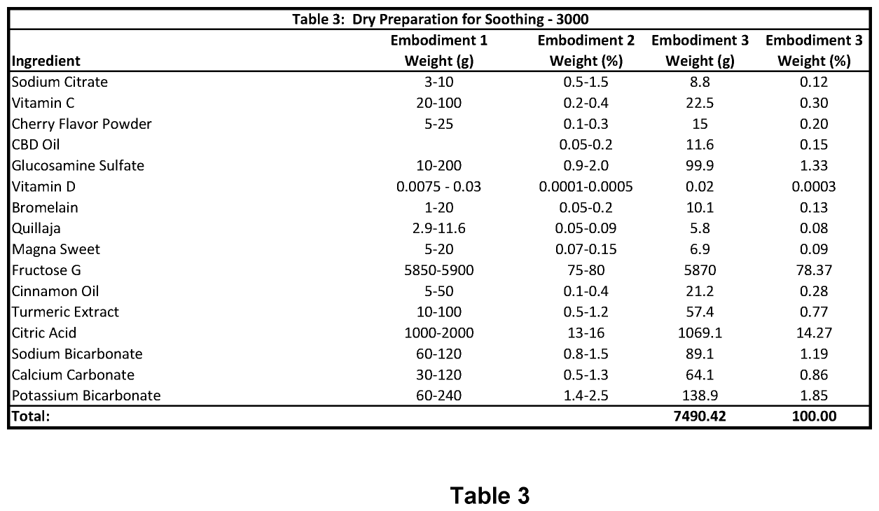

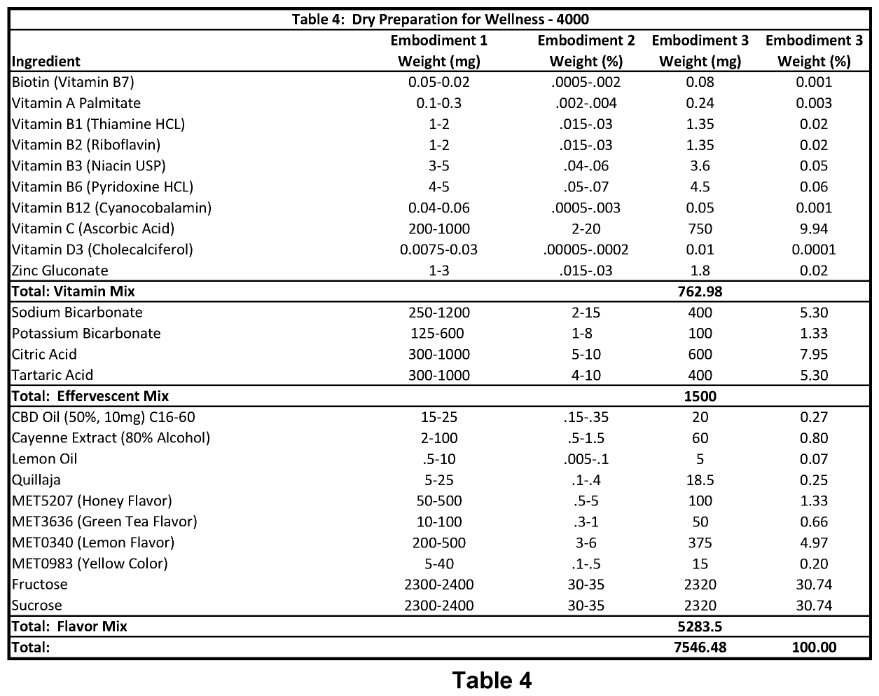

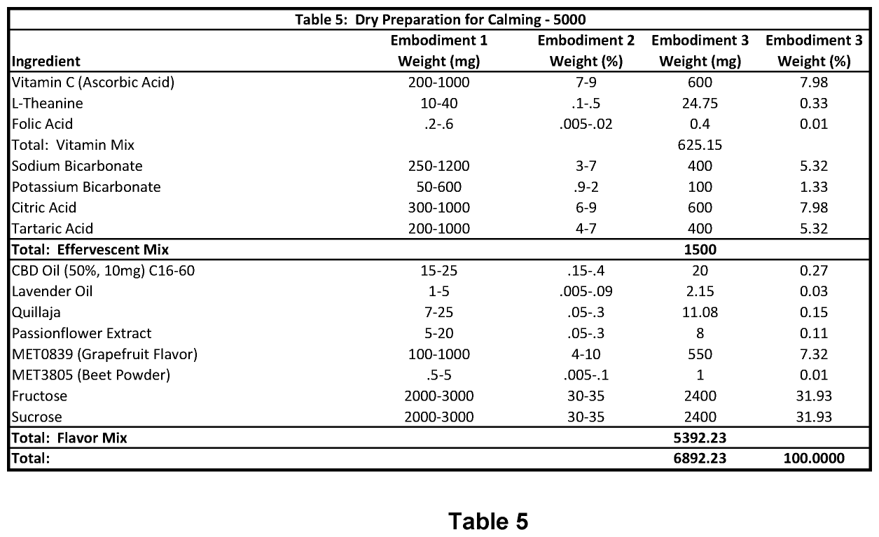

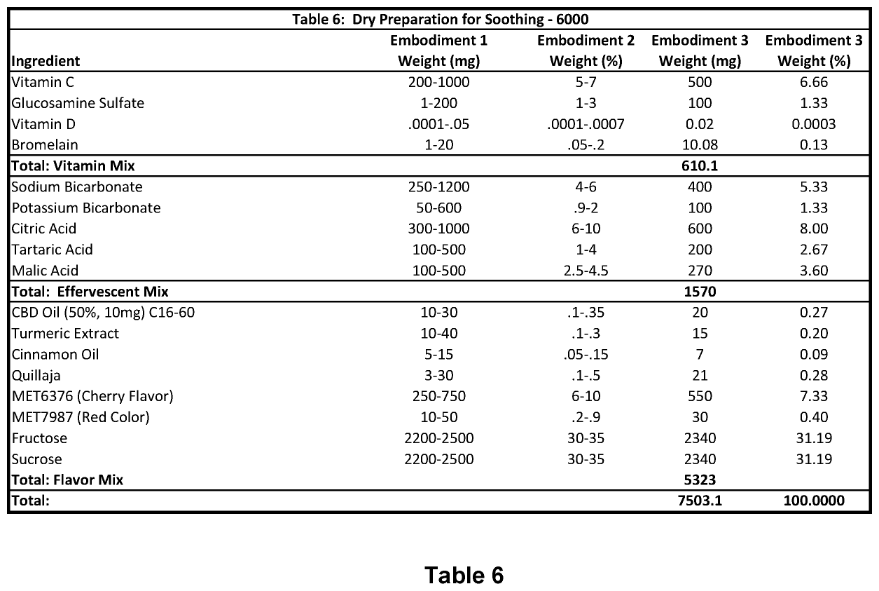

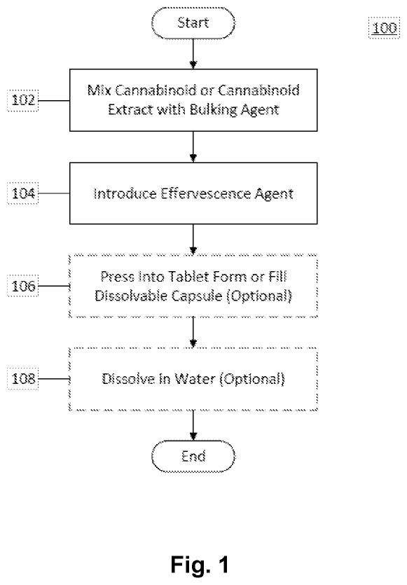

Turning now to FIG. 1, a method 100 of making a dry consumable preparation is now described. The method 100 may be used to make a dry preparation, such as the dry preparations for calming 1000, 5000 illustrated in Tables 1 or 5, the dry preparations for wellness 2000, 4000 illustrated in Tables 2 or 4, or the dry preparations for soothing 3000, 6000 illustrated in Tables 3 or 6. The method 100 may be implemented to produce about 7500 g of dry preparation 1000, 2000, 3000, 4000, 5000, 6000. Those skilled in the art will recognize that although the illustrated ingredients listings are scaled to about 7500 g of dry preparation, any scaling is suitable based off the weight in grams or milligrams, and such scaling is within the scope of this disclosure.

The method 100 of making a dry consumable preparation includes mixing 102 at least one of a cannabinoid or a cannabinoid extract containing one or more cannabinoids with a bulking agent. Mixing 102 may include mixing whereby the at least one of the cannabinoid or the cannabinoid extract containing one or more cannabinoids is plated onto the bulking agent. For example, those skilled in the art will recognize that the cannabinoid or cannabinoid extract containing one or more cannabinoids may be an oil. Mixing with the bulking agent and causing the oil to plate onto the bulking agent will cause the resulting mixture to behave like a dry product, because the oil is substantially encased. Mixing 102 may also include mixing any other oils included in the preparation and causing the oils to plate onto the bulking agent.

The method 100 also includes introducing 104 an effervescence agent to the mixture formed by the mixing 102. The effervescence agent may include sodium bicarbonate, potassium bicarbonate, and at least one acid. The at least one acid may include citric acid, tartaric acid, and/or malic acid. The effervescence agent may have a particular ratio of sodium bicarbonate to potassium bicarbonate acid. The ratio may be configured or selected to create a chemical pH buffering system at a targeted pH range when the dry consumable preparation is added to a targeted amount of water. Of note, a lower pH is associated with a less stable emulsion, with a pH of less than 2 being particularly unstable. At the same time, a lower pH results in less CO2 being released during effervescence, while a higher pH generates more CO2. The Applicants have determined that a pH of between 2 and 6, or between 4 and 5, may be suited to maximize the balance between nutrient uptake (nutrient uptake may be associated with effervescence) and a stable emulsion, as well as taste.

The method 100 may include pressing the mixture formed by the mixing 102 and the introducing 104 into a tablet form or filling the mixture into a dissolvable capsule 106.

The method 100 may include dissolving 108 the mixture formed by the mixing and the introducing 104 in a targeted amount of water.

The method 100 may produce an effervescence when combined with water. The powder formed by mixing 102 and introducing 104 may be configured to produce a stable emulsion in water which is maintained throughout the effervescence process. In some embodiments, the emulsion is maintained for several hours before degrading.

The method 100 may include providing enough CBD oil to deliver between about 5.0 g and 10. g, or about 7.5 g CBD across 1000 servings. Providing may include providing the CBD oil in a stainless-steel mixing container or any mixing container suitable for mixing the dry preparation. In some embodiments, a CBD oil concentration mix may be used. In some embodiments, a CBD oil concentration mix having about 64.5% CBD may be used. When using a 64.5% CBD concentration, 11.6 g of CBD oil should be used to provide 7.5 g of CBD across 1000 servings (7.5 g/0.645=11.6 g). Those skilled in the art will recognize that the remainder of the CBD oil concentration mixture may include fatty acids, flavonoids (flavor), terpenoids (aroma), carotenoids (color), waxes, chlorophyll, and other plant lipids. It is contemplated that the total concentration of CBD in each serving is less than 1%, or less than 0.5%, or less than 0.1%.

The method 100 may also include providing a bulking agent. The bulking agent may include or be a sweetener. The bulking agent may include fructose. Providing the bulking agent may include determining the amount of fructose or bulking agent to use. For example, the quantity of fructose may be dependent on the quantity of CBD oil used. For any given amount of CBD oil, the amount of fructose may be increased or decreased to reach a 7,500 g final powder weight. Fructose may be added as a bulking agent to adjust weight and to give the powder a sweet flavor. Additionally, or alternatively, the fructose may act as a masking agent to the bitter CBD flavor.

The method 100 may include providing a first group of ingredients. This first group of ingredients may include any essential oil desired to be included in the product, along with a botanical extract and/or Quillaja. For example, to produce the dry preparation for calming 1000 illustrated in Table 1, providing may include combining grapefruit and lavender essential oils along with Quillaja in a first mixing container and introducing this mix to the container having the bulking agent and CBD oil. In some embodiments, 25 g to 50 g, or about 32.1 g of grapefruit essential oil, an amount of lavender essential oil equal to the ratio of 15:1 grapefruit oil:lavender oil, and an amount of Quillaja equal to a ratio of about 1:1 to 1:4, or a ratio of about 1:2 Quillaja:oil are mixed together and then introduced to the container having the bulking agent and CBD oil.

To produce the dry preparation for wellness illustrated in Table 2, 10 g to 50 g, or about 35.0 g lemon essential oil, and an amount of orange essential oil equal to the ratio of about 0.5:1 to 1.5:1, or about 0.75:1 orange essential oil:lemon essential oil may be combined in a first mixing container and then introduced to the mix having the bulking agent and CBD oil.

To produce the dry preparation for soothing 3000 for soothing, 5 g to 50 g, or about 21.0 g cinnamon essential oil, 10 g to 100 g, or about 57.4 g turmeric extract, and an amount of Quillaja equal to a ratio of about 1:1 to 1:4, or a ratio of about 1:2 Quillaja:oil may be combined.

Providing the first group of ingredients may include providing ingredients that provide the user with nutraceutical properties associated with the ingredients.

Providing may include mixing the first group of ingredients, the bulking agent, and the CBD oil.

The method 100 may include allowing the mixture of CBD oil, bulking agent, and first group of ingredients to stand for a period of time. The period of time may be at least 2 minutes. The period of time may be between about 2 minutes and about 5 minutes. The period of time may be less than 10 minutes. The period of time may be a period of time suitable for allowing the mixture to homogenize. In some embodiments, the ingredients may be mixed with the CBD oil well enough for the CBD oil to homogenize.

The method 100 may include introducing the previously mixed ingredients to fructose. To prepare the dry preparation for calming illustrated in Table 1 or Table 5, the previously mixed ingredients may be introduced to between about 5,693 g and about 5,710 g, or about 5,703 g of fructose. To prepare the dry preparation for wellness illustrated in Table 2, the previously mixed ingredients may be introduced to between about 5,495 g and about 5,523 g, or about 5,517 g of fructose. To prepare the dry preparation for soothing 3000, the previously mixed ingredients may be introduced to between about 5,850 g and about 5,881 g, or about 5,870 g of fructose. Introducing may include introducing while mixing the fructose. Introducing may include introducing in a manner that prevents clumping. For example, the fructose may be in a mixing container set on a low setting, and the previously mixed ingredients may be slowly introduced over a period of several minutes.

Introducing may include determining whether clumps are forming. If so, introducing may include pausing the introducing to scrape the container and break down clumps. This may occur about every 60 seconds. The mixing device or container may be powered off to allow for a user to scrape an interior of the mixing bowl to break up clumps that have formed. Introducing may include introducing into a vertical cutter mixer (VCM).

The VCM may be used at a low mixing speed to thoroughly combine the ingredients, and it may be necessary to continue to power off or pause the device in order to scrape the sides or other portions of the mixing device to break up clumps every 30-60 seconds. Mixing and scraping occurs until the powder is homogenous, at which point the remainder of the dry ingredients may be added to the mixture.

The method 100 may include introducing the remaining ingredients. Introducing the remaining ingredients may include mixing thoroughly. To provide dry preparation for calming illustrated in Table 1, the remaining ingredients may include about 3 g to 10 g or about 3.7 g of sodium citrate, about 20 g to 100 g or about 22.5 g of Vitamin C, about 20 g to 50 g or about 24.9 g of Coenzyme Q10, about 5 g to 10 g or about 8.5 g of Magna Sweet, about 20 g to 50 g or about 24.7 g of L-theanine, about 10 g to 20 g or about 15.0 g of passionflower flavor powder, about 1,300 g to 1,400 g or about 1,327 g of citric acid, about 60 g to 120 g or about 66.4 g of sodium bicarbonate, an amount of calcium carbonate equal to the ratio of 1:1 to 2:1, or about 1.2:1 calcium carbonate:sodium bicarbonate, and an amount of potassium bicarbonate equal to the ratio of 2:1 to 3:1, or about 2.6:1 potassium bicarbonate:sodium bicarbonate, to the container or VCM bowl.

To produce the dry preparation for wellness illustrated in Table 2, the remaining ingredients may include about 1 g to 50 g or about 11.9 g of cayenne extract, which may be added and mixed prior to adding the remainder of the dry ingredients including about 0.1 g to 0.3 g or about 0.2 g Vitamin A, about 0.0075 g to 0.03 g or about 0.015 g Vitamin D3, about 4 g to 5 g or about 4.5 g Vitamin B6, about 3 g to 5 g or about 3.6 g Vitamin B3, about 1 g to 2 g or about 1.3 g Vitamin B2, about 1 g to 2 g or about 1.3 g Vitamin B 1, about 0.04 g to 0.05 g or about 0.046 g Vitamin B12, about 20 g to 100 g or about 22.5 g Vitamin C, about 1 g to 2 g or about 1.8 g zinc gluconate, about 0.05 g to 0.2 g or about 0.1 g Biotin, about 60 g to 120 g or about 117.0 g sodium bicarbonate, an amount of calcium carbonate equal to the ratio of about 0.5:1 to 1:1 or about 0.7:1 calcium carbonate:sodium bicarbonate, an amount of potassium bicarbonate equal to the ratio of about 1:5 to 3:1 or about 2:1 potassium bicarbonate:sodium bicarbonate, and 1,000 g to 2,000 g or about 1,338.1 g citric acid.

To prepare the Table 3 preparation, the remaining ingredients may include about 3 g to 10 g or about 8.8 g sodium citrate, about 20 g to 100 g or about 22.5 g Vitamin C, about 5 g to 25 g or about 15.0 g Cherry flavor powder, about 10 g to 200 g or about 99.9 g glucosamine sulfate, about 0.0075 g to 0.03 g or about 0.02 g Vitamin D, about 1 g to 20 g or about 10.1 g bromelain, about 5 g to 20 g or about 6.9 g Magna Sweet, about 1,000 g to 2,000 g citric acid or about 1,069.1 g citric acid, about 60 g to 120 g or about 89.1 g sodium bicarbonate, an amount of calcium carbonate equal to the ratio of about 0.5:1 to 1:1 or about 0.7:1 calcium carbonate:sodium bicarbonate, and an amount of potassium bicarbonate equal to the ratio of about 1:1 to 2:1 or about 1.5:1 potassium bicarbonate:sodium bicarbonate.

Introducing any ingredients may include introducing enough to provide a therapeutically effective amount of the ingredients, such as to provide the user with the nutraceutical properties and/or flavoring associated with each ingredient.

For example, a high level of Vitamin D may reduce chronic pain and inflammation from fibromyalgia. Furthermore, Magna Sweet includes a super sugar ingredient, made up of glycyrrhizic acid which is derived from licorice root. The Magna Sweet may also reduce the bitterness of the CBD, providing a prolonged sweetness on the tongue. Introducing may include mixing thoroughly, in a manner similar to that described with reference to introducing. For example, the mixing device may be powered on to a low mixing setting and allowed to thoroughly combine the ingredients, with the user or device periodically scraping the sides of the mixing device and breaking up any clumps as necessary until the powder is homogenous.

As previously described, the dry preparation 1000, 2000, 3000, 4000, 5000, 6000 may include a powder mixture having one or more cannabinoids, cannabinoid extract containing one or more cannabinoids, sucrose, fructose, Quillaja, one or more herbal extracts, one or more nutrient vitamins and minerals, and an effervescence mixture, wherein the powder composition forms a stable, effervescent emulsion when mixed with water.

The one or more herbal extracts may include one or more of Cayenne extract, Lemon oil, Turmeric extract, cinnamon oil, bromelain, lavender oil, tart cherry, and passion-flower extract. Additionally, or alternatively, the one or more herbal extracts may be an essential oil. Some non-limiting examples of essential oils that may be used in the dry preparation include bergamot, clove, marjoram, thyme, lime, fennel, peppermint, and citrus essential oils.

The one or more vitamins and minerals may include L-theanine, Vitamin C, Folic Acid, Vitamin D, Glucosamine Sulfate, Biotin, Vitamins A, B1, B2, B3, B6, B12, and zinc gluconate.

In some embodiments, the effervescence mixture includes sodium bicarbonate, potassium bicarbonate, citric acid, tartaric (taste quality) acid, and malic acid. Further, the ratio of sodium bicarbonate to potassium bicarbonate to acid (e.g., citric acid, tartaric acid, malic acid, or any other suitable acid) when added to water creates a chemical pH buffering system at a target pH range. The targeted pH range may be between about 2 and about 6. For instance, the targeted pH range may be between about 3 and 4. This chemical pH buffer system may resist changes to the target pH band of the effervescent powder/water solution. For example, the addition of excess acid, such as from mixing the effervescent powder with a fruit juice for example, would result in a small change in the pH of the effervescent powder/juice solution from the target pH band. The chemical pH buffering system provides the advantage of maintaining the pH of the effervescent powder water solution within a narrow target pH band to promote stability of the emulsion.

In addition to establishing the chemical pH buffering system, the effervescence created by the effervescent powder may impart advantages in palatability and delivery and absorption of the cannabinoid mixture and other nutrients within the formula. The presence of the carbon dioxide bubbles has been shown to enhance absorption of vitamins and minerals in the digestive tract. Further, effervescence has been shown to reduce stomach upset. Additionally, effervescence may be used to mask the bitter taste of some of the bitter flavonoids and biomolecules that may be present in cannabinoid oils and isolates, such as piperidine, for example.

The cannabinoid extract may be a cannabinoid oil or cannabinoid isolate. Further, the cannabinoid extract may be in liquid, crystalline, or liquid crystalline form.

As illustrated in Table 4, a general wellness formula 4000 may include one or more of: a complex of B Vitamins, Vitamin C, Vitamin D3, zinc gluconate, Vitamin A, Cayenne Pepper extract, Lemon Oil, and Cannabidiol, which may serve to promote immune system health (Vitamin C), improve energy (B Vitamin complex, zinc, Cayenne Pepper extract, and Lemon Oil), reduce inflammation (Cannabidiol and Cayenne Pepper extract), support bone health (Vitamin D3) and support vision and reduce oxidative stress (Vitamin A).

As illustrated in Table 5, a calm formula may include Vitamin C, L-theanine, Folic Acid, lavender oil, passionflower extract, and cannabidiol to promote a calm mood (L-theanine, Cannabidiol, lavender oil, and passionflower extract) and promote more efficient cellular energy metabolism which has been shown to aid in overcoming depression.

As illustrated in Table 6, a soothe formula may include Vitamin C, Vitamin D, Bromelain, Glucosamine sulfate, and Cannabidiol to reduce inflammation in the body (Cannabidiol and Bromelain) and support joint health (Vitamin D and Glucosamine Sulfate). The MET 7987 (Red Color) provides a source of cyanidin which is a free radical scavenger and has antioxidant properties.

The effervescence is achieved through the reaction of sodium bicarbonate, potassium bicarbonate, citric acid and tartaric acid in water. This reaction releases carbon dioxide. It will be appreciated that coated versions of these ingredients or other suitable acids and bases may be used to prolong effervescence. Further, the tartaric acid and malic acid are used to improve flavor and reduce clumping due to these acids being less hygroscopic than citric acid. Acids have different taste profiles and are used to modify flavor profile. Citric acid delivers a “burst” of tart flavor, similar to fresh fruit. Tartaric acid delivers an extremely tart taste compared to citric acid. Lactic acid delivers an acrid profile. Ascorbic acid is described as sour and tart. Oxalic acid is described as pleasantly sour. Tannic acid delivers zesty tartness and bitterness. Phosphoric acid is described as tangy and sour. Malic acid delivers smooth, dry tartness.

In some embodiments, a ratio of sucrose to fructose of about 1:1 by weight is provided in the dry preparation 1000, 2000, 3000, 4000, 5000, 6000. This ratio creates an adequately sweet (but not overly sweet) emulsion consumption which mediates the hygroscopicity of the fructose and prolongs the shelf-life of the product.

In some cases, the dry consumable preparation, also referred to as cannabinoid preparation, described in this disclosure may be configured to retain at least 85% of a stated cannabinoid potency for the cannabinoid preparation two years after producing the cannabinoid preparation when stored at standard environmental conditions. In some cases, standard environmental conditions, as defined by the National Institute of Standards, is 20 degrees Celsius (+/−4 degrees Celsius) temperature and 1 atmosphere (+/−0.2 atmosphere) pressure. Further, potency may be defined as the amount of cannabinoid preparation needed to achieve the intended result of the product (calming effect, increased energy, etc.). In some circumstances, cannabinoid potency may be maintained by one or more of the following factors: a low oxygen environment within the packaging, the presence of natural antioxidant properties of the botanical extracts contained within the formulation, the adsorbent properties of the plating agent, and choice of encapsulation techniques.

Some non-limiting examples of botanical extracts may include cannabis, passionflower, grapefruit, lavender, green tea, lemon, orange, cayenne, cherry, cinnamon, and turmeric extracts. In some cases, one or more of the botanical extracts listed above may contain natural antioxidants, including cannaflavins, flavonoids, carotenoids, terpenoids, alkaloids, curcuminoid, catechins, and/or polyphenols. In some cases, antioxidants in the dry consumable preparation may also serve to enhance the shelf life of the preparation. As used herein, the term antioxidants may refer to compounds, such as polyphenols, that inhibit oxidation. Oxidation is a chemical reaction that produces one or more free radicals. In some cases, oxidation may lead to a chain reaction where numerous free radicals may be formed, which may cause damage to the cells of organisms. In some examples, antioxidants may terminate the chain reactions that produce the free radicals, which may not only enhance the shelf stability of the dry consumable preparation but also inhibit the formation of free radicals in the body. In some cases, the formation of free radicals may be linked to certain types of cancers and/or tumors in humans.

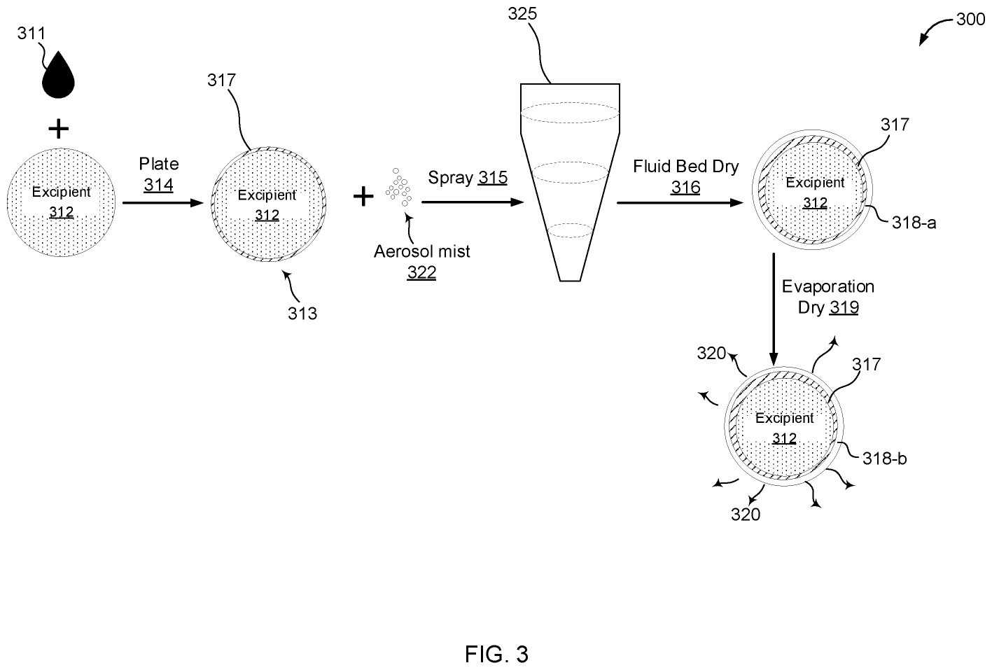

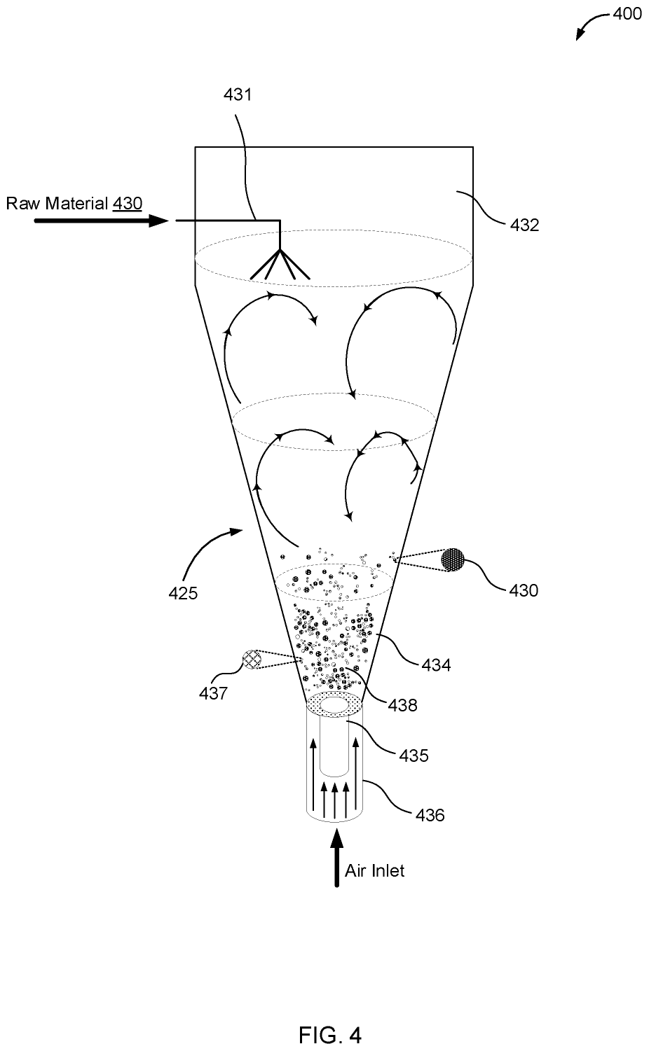

With respect to the plating agent helping to maintain cannabinoid potency, as described herein, it is contemplated that in using an adsorbant plating agent (also referred to herein as plating or a plating layer), less cannabinoid oil will be exposed to the environment, thereby protecting loss that oil from oxidation and UV degradation. In some cases, encapsulation techniques can include fluid bed drying (described later in relation to FIGS. 3 and 4), granulation, and/or mechanical mixing. In some examples, mechanical mixing may include the use of a v-blender, ribbon blender, planetary mixer, or high shear mixer, to name a few non-limiting examples.

In addition to shelf stability, the dry consumable preparation may be configured to remain safe for human consumption at least 2 years after production and packaging, for instance, when stored under standard environmental conditions. In some cases, safety may comprise microbial safety, which may be defined as one or more of: Escherichia Coli (E. Coli) Colony-forming units (CFU) under 1 per gram (i.e., E. Coli<1 CFU/g), Salmonella<1 CFU/g, total yeast and mold<10,000 CFU/g, water activity<0.85, to name a few non-limiting examples.

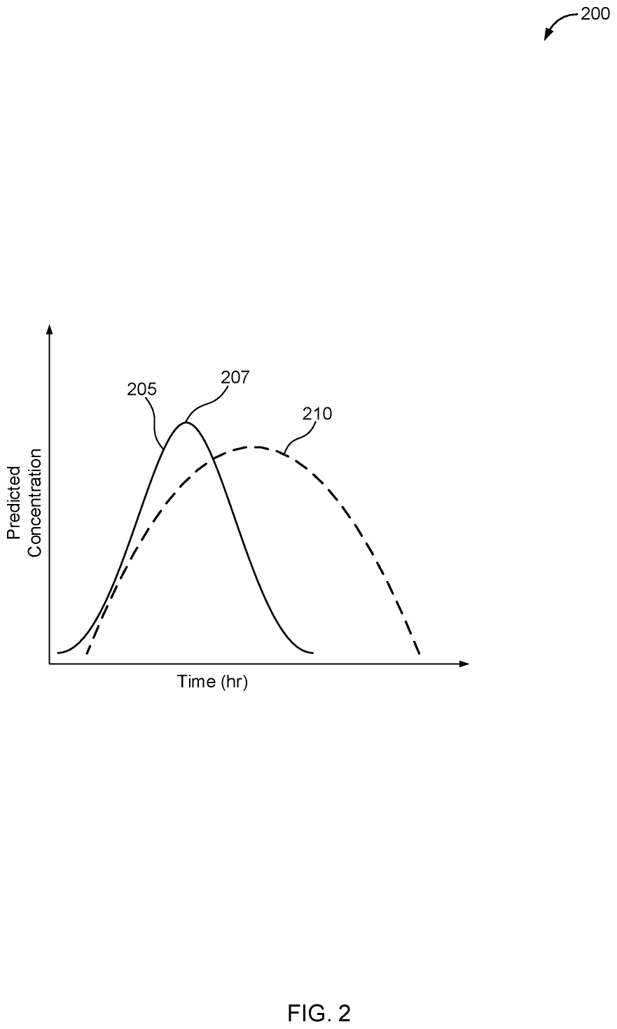

FIG. 2 illustrates a graph 200 comparing the predicted concentration of a sustained release formulation with that of an immediate release formulation as a function of time. According to aspects of this disclosure, the dry consumable preparation may be an example of an immediate release formulation that provides a consumer with a greater degree of control over the effects of the preparation upon ingestion. In some aspects, the degree of control may refer to a level of predictability of the effects of the preparation from ingestion until wear off. In other words, as compared to a sustained formulation that exhibits a combination of sigmoidal and quadratic pharmacokinetic curves 210, the dry preparation of the present disclosure may exhibit a conventional bell pharmacokinetic curve 205. The combination of sigmoidal and quadratic curve 210 and the bell pharmacokinetic curve 205, respectively, relate to a predicted concentration (e.g., plasma drug concentration) in a consumer's body from onset until offset of effects. In one example, the pharmacokinetics of the dry preparation of this disclosure comprise a bell shape, with onset and offset (or decline) within 4 to 6 hours. This may be fundamentally different than a sustained release formulation that provides the combination of the sigmoidal and quadratic pharmacokinetic curves 210 with a substantially longer decline duration. For instance, the combination of sigmoidal and quadratic pharmacokinetic curves 210 may span a duration of anywhere between 8 to 10 hours (i.e., from onset to offset). By reducing the time duration between onset and offset, the effects of the dry consumable preparation may be more predictable to a consumer. In other words, a consumer may be able to predict the onset and offset of a preparation exhibiting a conventional bell pharmacokinetic curve more accurately, than if the preparation exhibited a combination of sigmoidal and quadratic pharmacokinetic curves.

The conventional bell-shaped curve 205 displays a shallow curve portion during an initial dissolution time as the tablet dissolves. Once the tablet has dissolved, the drug concentration curve steepens dramatically. As the drug is metabolized, the drug concentration curve flattens again, creating the top or peak 207 of the bell-shaped curve, then dramatically falls off. The concentration curve flattens as the remaining drug is metabolized. The beginning of a sustained release pharmacokinetic curve 210 is similar to the conventional bell-shaped curve except for the duration of time of initial dissolution is extended and exaggerates the sigmoidal shape. A sustained release formula reduces the dramatic flattening of a conventional bell-shaped curve by reducing the rate at which the drug is released, thereby increasing the time needed to reach reduced drug concentration levels. A sustained release formula exaggerates the x-axis time factor needed for initial dissolution and subsequent drug release thereby broadening the curve to create a sigmoidal curve as the tablet dissolves and a quadratic curve as the drug is slowly released.

In one example, a peak 207 of the conventional bell pharmacokinetic curve 205 may occur around 2-3 hours after ingestion or oral conventional dosing of the dry consumable preparation. Further, the effects of the preparation may wear off around 2-3 hours after the peak 207. Said another way, the onset and offset of the effects of the dry consumable preparation may occur in a conventional bell curve fashion. In this example, the effects may be realized (i.e., the “onset” of the physiological effects my occur) within 1 hour and may offset after 5 hours of oral dosing. The conventional bell shape of the pharmacokinetic curve may allow the consumer to predict the effect time more easily. One effect of a Calm formulation is “enhanced relaxation”. For Soothe formulation, the effect may be to “ease aches”. For General Wellness, the effect may comprise “increased vitality”.

FIG. 3 illustrates a process flow 300 for producing a cannabinoid preparation according to an embodiment of the disclosure. In some cases, process flow 300 implements one or more aspects of the figures described herein, including at least FIG. 4. As shown, process flow 300 may begin with one or more excipient particles 312 and oil 311, wherein the oil 311 may comprise cannabinoid oil. In some embodiments, the one or more excipient particles 312 may comprise an oil adsorbent agent and an outer surface having one or more pores and capillaries. Some non-limiting examples of the oil adsorbent agent may comprise at least one of maltodextrin, cyclodextrin, and a polysaccharide gum. Further, the excipient particles 312 may comprise one of a carbohydrate sweetener, a water-soluble powdered flavor, and a water-soluble powdered color. In some cases, the carbohydrate sweetener comprises at least one of sucrose, fructose, glucose, corn syrup, corn syrup solids, lactose, honey, agave nectar, allulose, maltodextrin, tapioca syrup, digestion resistant maltodextrin, trehalose, galactose, and starch. In some cases, the carbohydrate sweetener comprises about a 1:1 ratio of sucrose and fructose. Alternatively, the ratio of the sucrose to fructose is about 1:3 (i.e., a third ratio).

As known to a person of skill in the art, adsorption may refer to the process of adhesion (e.g., molecular adhesion) of molecules of a liquid or gas onto the surface of a solid particle. In some aspects, adsorption is a surface phenomenon wherein molecules simply attach to the surface of an adsorbent. In contrast, absorption is a bulk phenomenon wherein molecules of an absorbate enter into an absorbent. According to aspects of this disclosure, plating 314 may involve adsorption onto the exterior surface of a particle, as with a crystalline sugar for instance. Furthermore, the plating 314 may also involve absorption onto and into a particle, as with silica for instance.

In some cases, the porous structure of the excipient particles 312 may be crystalline in nature (e.g., fracture patterns). In some other cases, the porous structure of the excipient particles 312 may resemble a golf ball (e.g., with pock marks or surface indentations), a sponge (e.g., having capillaries that go through the center), an asteroid (e.g., dented or pock marked), to name a few non-limiting examples. The porous structure of the excipient particles 312 may serve to provide a three-dimensional (3D) framework onto which the cannabinoid oil will adhere during plating 314. In some circumstances, oil may be more easily adsorbed onto a particle comprising large void volumes and/or pore sizes as compared to a particle having small void volumes and/or pore sizes. Said another way, large void volumes and/or pore sizes in a particle may serve to stimulate adsorption of oil since porosity is directly related to surface area. Further, a higher surface area of a porous particle may enable oil to be adsorbed at a higher rate or more easily than if the porous particle had a lower surface area (i.e., less exposed surface area). One non-limiting example of a porous particle with a low surface area may include a cubic crystal. Contrastingly, a porous sphere may be an example of a porous particle with a high surface area. It should be noted that, in the above examples, the cubic crystal and the porous sphere may have similar or substantially similar dimensions (e.g., the height or width of the cubic crystal may be roughly equal to the diameter of the sphere, or the sphere and cubic crystal may be similar in volume). In some cases, the particle structure of the excipient particles may be varied via adjustment of heating rate, heating temperature, drying rate, or drying temperature, to name a few non-limiting examples.

In some cases, the porous structure of silica may be characterized as a face centered cubic packed structure with octahedral and/or tetrahedral voids. In one example, Zeofree 5161 Silica may absorb 200 cubic centimeters (cc) of oil per 100 grams. In another example, food starch, such as Ingredion NZorbit 2144 maltodextrin may absorb 70 cc oil per 100 grams. In some cases, the pore structure of food starch may be round. Additionally, or alternatively, the pore structure of food starch may comprise a polygonal granular shape with internal cavities extending from the surface to the interior of the particle.

In some circumstances, higher levels of adsorption may facilitate in enhancing powder flow during packaging, dissolution during consumption, and/or homogenous potency, to name a few non-limiting examples. For instance, a higher level of adsorption may also mean that a higher proportion of oil in an excipient particle 312 is bound oil as compared to free oil. As used herein, the term free oil refers to the surface oil (i.e., oil on the exterior surface area of an excipient particle 312), while bound oil refers to the oil that is on the interior surface of the excipient particle 312 (e.g., retained in the pores). In some cases, the surface oil to total oil ratio provides the percentage of exposed oil on the surface of the excipient particle 312 that is free/available to contact the environment. A higher level of adsorption implies that there is a minimal amount of surface oil or free oil to obstruct the flow of powder, which may allow the excipient particles 312 in the powder to also flow freely. As noted above, the excipient particles 312 may comprise a material with any porous structure. If the excipient has low oil absorbency (e.g., below 10 cc oil per 100 grams), the excipient particles 312 may have a high free surface oil:total oil ratio. To alleviate the impact on flow characteristics and/or shelf life, the excipient particle 312 can be encapsulated with an exterior protective shell, further described below.

In some cases, higher absorption also results in a lower proportion of the surface oil that is exposed to the environment. With regards to powders, flow characteristics may be affected by free surface oil content. For instance, a higher proportion of free surface oil content can cause agglomerations of particles that lead to clumping and inhibition of smooth flow characteristics. In some examples, agglomeration techniques may be utilized to optimize one or more properties, such as density, flowability, reconstitution time, etc., of powdered preparations. In some cases, a lower reconstitution time may be linked to the creation of inter-particles pores available for capillary rise, where the capillary rise may relate to the wetting properties of the solid material. In some cases, the dissolution time for a porous powder may be lower than a crystallized powder. A faster dissolution time may serve to enhance consumer experience (e.g., powder is more easily dissolved, less clumping, etc.).

In some embodiments, the oil plated excipient particles 313 may include a core or inner layer (i.e.., excipient particles 312) surrounded by an oil plating layer 317. Further, the oil plate excipient particles 313 may comprise from about 0.5% to about 1.5% of their weight in oil 311 (e.g., a cannabinoid oil). In some cases, the density of a physical bond between the oil 311 and the excipient particles 312 may affect a level of contact of the oil plated excipient particles 313 with environmental oxygen, release rate of oil from the oil plated excipient particles 313, etc. In some cases, a denser physical bond between the oil 311 and the excipient particles 312 may serve to decrease the contact between the oil plated excipient particles 313 and oxygen, which may in turn allow less oil to be released from the oil plated excipient particles 313 over time. A high number of pores on excipient particles 312 may also enhance the retention of oil due to the lower level of contact between adjacent oil plated excipient particles 313.

The oil plated particles 313 can be agglomerated or lecithinized to increase particle size, ease production, speed dissolution when added to the desired amount of water, increase cannabinoid bioavailability, to name a few non-limiting examples. In some examples, lecithinization may refer to a process of adding lecithin to the outer surface of agglomerated particles to maintain rheological properties, such as flow and rapid dissolution. The phospholipid content within the lecithin serves to emulsify cannabinoids once the powder is added to the desired amount of water. As the outer surface of the lecithin begins to dissolve in the water, the phospholipids may interact with the cannabinoids to form a stable emulsion.

In some cases, particles having a size less than 1000 um may be referred to as powders, where the size may refer to a largest dimension of the particle. For instance, the size may refer to a diameter (e.g., of a sphere), a longest diagonal of a cuboid, a diagonal of a cube, to name a few non-limiting examples. In other cases, the size may refer to the radius of a sphere, or a length/width/height of a cube/cuboid. In some circumstances, particles having a size under a threshold may require up to 2% lecithinization, whereas enhanced wetting properties of larger particles (i.e., above a size threshold) may be accomplished at up to 0.25% lecithinization.

Particles having sizes ranging from about 100 micron to about 1000 micron, for instance, from 100 to 500 micron, or 150 to 250 micron, may provide optimal flow characteristics. In such cases, upon agglomeration, the target particle size may be about 200 micron.

In some embodiments, the plated powder comprising the plurality of oil plated excipient particles 313 can be further processed to add a complete protective outer shell that encapsulates each of the oil plated excipient particles. In some embodiments, the protective outer shell comprises a water-soluble material deposited on an exterior surface area of the oil plated excipient particles 313 (e.g., water soluble material is sprayed 315 in the form of an aerosol mist 322). In some cases, a fluid bed dryer 325 may be utilized to deposit protective outer shell 318-a, further described in relation to FIG. 4. In some cases, the outer shell 318-a can serve as one or more of an oxidative barrier, flow aid, bulking agent, dissolution agent, and a carrier of other active ingredients, wherein the active ingredients comprise one or more of dietary mineral content and an effervescence agent. Additionally, or alternatively, the active ingredients may comprise carbonate of sodium, potassium and calcium, which may facilitate in delivering dietary mineral content or, when combined with citric, tartaric, lactic, acetic, benzoic, ascorbic, oxalic, tannic, phosphoric, or malic acid may serve to produce an effervescent effect.

As shown, the oil plated excipient particles 313 may be fluid bed dried 316 with an encapsulant, such as sodium bicarbonate, in fluid bed dryer 325. For instance, a water based or aqueous solution comprising sodium bicarbonate may be sprayed 315 on to the oil plated excipient particles 313 as the aerosol mist 322 in a chamber of the fluid bed dryer 325. As illustrated, after processing the oil plated excipient particles 313 in the fluid bed dryer 325, the particles may now comprise one or more layers, including the core (i.e., excipient 312), the oil plating layer 317, and the exterior shell 318-a. It should be noted that, the exterior shell 318-a is a wetted surface on the oil plated excipient particle 313. In some embodiments, this wetted surface may be dried 319 (e.g., evaporation dried). Evaporation drying may be used to dry the exterior wetted surface (i.e., exterior shell 318-a) of the oil plated excipient particle 313 to release moisture 320 prior to packaging and shipping to consumers. Once evaporation drying 319 is complete, the dry consumable preparation comprising excipient core 312, oil plating layer 317, and dried exterior shell 318-b is ready for packaging and consumption.

In some embodiments, the dry cannabinoid preparation may be configured to retain at least 85% of a stated cannabinoid potency for the cannabinoid preparation two years after producing the cannabinoid preparation. Additionally, or alternatively, the cannabinoid preparation may be configured to retain its effervescence properties over a 2-year shelf life, by, for instance, minimizing water vapor transmission across the packaging. In some cases, as water concentration builds up in the atmosphere/air within the packaging (e.g., due to hygroscopicity), effervescent agents may begin to react, which may adversely impact the desired effectiveness and/or sensory experience of the preparation. To mitigate such issues, the cannabinoid preparation of the current disclosure may be stored in packaging wherein the headspace of the packaging is purged with an inert gas, such as nitrogen. This purging with the inert gas may serve to replace the oxygen and/or relative humidity with a stable “cap” of nitrogen in the headspace of the packaging. Besides effervescence and cannabinoid potency, the cannabinoid preparation may also be configured to retain a threshold amount of vitamins, flavor, color, minerals, nutritional profile, etc., over the 2-year shelf life. It should be noted that, the 2-year shelf life is merely an example, and a longer shelf life may be contemplated in different embodiments.

FIG. 4 illustrates an embodiment of a system 400 configured to be used for encapsulation. As shown, system 400 comprises a fluid bed dryer 425, the fluid bed dryer 425 comprising an input for raw material 430, a spray nozzle 431, an air inlet pipe 436, an expansion chamber 432, a drying chamber 434, and a product discharge pipe 435. As seen, the product discharge pipe 435 may be substantially surrounded by the air inlet pipe 436 or may be positioned within the air inlet pipe 436. In some cases, the product discharge pipe 435 and the air inlet pipe 436 may be concentric. In some examples, fluid bed dryer 425 may be similar or substantially similar to the fluid bed dryer 325 previously described in relation to FIG. 3.

In some cases, encapsulation techniques may include one or more of fluid bed drying, granulation, and mechanical mixing, where mechanical mixing may include the use of a v-blender, ribbon blender, planetary mixer, and/or high shear mixer. In some embodiments, fluid bed drying may be employed to provide a protective shell on the exterior of a particle, such as an oil plated excipient particle (shown as oil plated excipient particle 313 in FIG. 3). In some cases, the protective shell (also referred to herein as an encapsulant) can be an aqueous solution containing a wide variety of water-soluble constituents that when dehydrated may deposit a continuous layer (e.g., dried exterior shell 318-b in FIG. 3) across the surface of the plated particle (e.g., particle composed of excipient core 312 and oil plating layer 317 in FIG. 3). In some circumstances, this protective shell layer can serve to deliver functional ingredients, including carbonate of sodium, potassium, and calcium, where the carbonate can combine with food acids to produce an effervescent effect. In some cases, the effervescent effect may serve to decrease the dissolution time.

In some cases, the raw material 430 may comprise wet material including a solution (e.g., aqueous solution of sodium carbonate or sodium bicarbonate, to name two non-limiting examples) and the oil plated excipient particles (e.g., oil plated excipient particle 313 in FIG. 3). In other cases, the raw material 430 may comprise wet material including a solution of only the constituents of the exterior protective shell (e.g., an aqueous solution of sodium carbonate or sodium bicarbonate, to name two non-limiting examples) that is sprayed from above onto a fluidized bed of oil plated excipient particles 437. The spray nozzle 431 may be used to spray the wet raw material in an expansion chamber 432 positioned near a top of the fluid bed dryer 425. In some cases, hot and/or dry air may enter the fluid bed dryer via the air inlet pipe 436. The air inlet pipe 436 may be positioned near a bottom of the fluid bed dryer below the drying chamber 434. In some cases, the hot and/or dry air may mix with the wet raw material 430 sprayed from the spray nozzle 431 and cause the wet particles (i.e., oil plated excipient particles with a wetted surface) to agglomerate in a lower end of the fluid bed dryer 425 (e.g., in the drying chamber 434 of the fluid bed dryer 425) into granules of a desired size. In other cases, the hot and/or dry air may mix with a bed of oil plated excipient particles 437 in such a way to create a fluidized bed 438 in the lower portion of the drying chamber. In some cases, the granules (or dry particles) may be of a uniform or substantially uniform size, for instance, if the oil plated excipient particles are uniform or substantially uniform. Further, the thickness of the dry exterior shell of the dry particles may be based on a variety of factors, such as the moisture content of the aqueous solution, temperature of incoming hot/dry air, drying time, size of oil plated excipient particles, etc. The oil plated excipient particles, ranging in diameter between 20 microns and 200 microns for each particle and preferably between 50-155 microns, can enter the fluid bed dryer 425. As the encapsulant accumulates on the particles, the diameter of each particle will increase up to 1.5 times, to between 30 microns and 300 microns and preferably between 150-250 microns. Particles will begin to adhere to each other and form multiple particle-containing granules. The granules will range in size from 250 um to 1000 um, depending on required dissolution and flow properties. The oil-plated particle may be part of the wet material 430 or may be a standalone powder 437 that is loaded into the bottom of the dryer to be mixed with hwqq211ot air to form a fluidized bed 438 onto which the raw material 430 (e.g., aqueous solution of sodium carbonate or sodium bicarbonate) is sprayed. In some embodiments, the dry particles having an excipient core, a plated oil layer, and a dry exterior shell may be collected from the fluid bed dryer 425 via the product discharge pipe 435.

In some cases, the encapsulated particles collected via the product discharge pipe 435 are now ready for packaging and consumption. In some cases, the consumption may comprise adding the encapsulated particles (e.g., in a powdered beverage form) to a liquid medium, where the liquid medium may be selected from a group consisting of water, soda, juice, milk, to name a few non-limiting examples. In some cases, the functional ingredients of the encapsulated particles (i.e., in the exterior shell layer surrounded the oil plated particle) may be configured to combine with food acids to produce an effervescent effect. Further, the encapsulated particles may be configured to dissolve within the liquid medium to produce a homogenous or substantially homogenous solution.

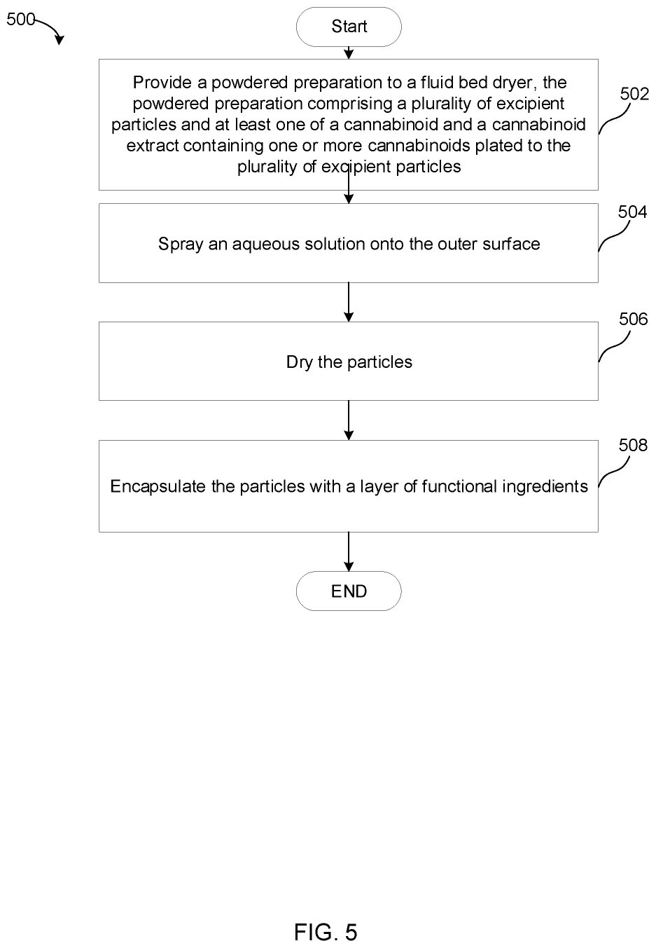

FIG. 5 illustrates a method 500 of producing a cannabinoid preparation according to an embodiment of the disclosure. In some cases, method 500 implements one or more aspects of the figures described herein, including FIGS. 3 and 4.

In some cases, the method 500 comprises providing 502 a powdered preparation to a fluid bed dryer, wherein the powdered preparation comprises a plurality of excipient particles and at least one of a cannabinoid and a cannabinoid extract containing one or more cannabinoids plated to the plurality of excipient particles. In some embodiments, the plurality of excipient particles comprise an oil adsorbent agent and an outer surface having one or more pores and capillaries. Further, the at least one of the cannabinoid or the cannabinoid extract containing one or more cannabinoids may be plated to the outer surface, pores, and/or capillaries of the plurality of excipient particles.

Further, the method 500 comprises spraying 504 an aqueous solution onto the outer surface of the plated excipient particles (e.g., shown as oil plated excipient particle 313 in FIG. 3 comprising an excipient core 312 and oil plating layer 317). In some cases, the spraying may be performed using a spray nozzle, such as the spray nozzle 431 described in relation to FIG. 4. In some cases, the aqueous solution may comprise a water-based solution of carbonate of sodium, potassium, and/or calcium, which may serve to impart an effervescent effect when the cannabinoid preparation is dissolved in a liquid, such as water.

In some cases, the method 500 may further comprise: drying 506 the oil plated excipient particles having a wetted surface or outer layer of the aqueous solution. In some cases, the drying 506 may comprise evaporation drying or fluid bed drying.

In some cases, the method 500 may further comprise encapsulating 508 the particles with a layer of functional ingredients, the functional ingredients selected from a group consisting of effervescent agents, vitamins, acidulants, sweeteners, flavors, and botanical extracts. In one example, the functional ingredients may comprise a water-soluble starch. In another example, the functional ingredients may include carbonate salts of sodium, potassium, and/or calcium. As previously described, the carbonate salts may combine with food acids to produce an effervescent effect, where the effervescent effect may serve to decrease the dissolution time (i.e., time it takes for the powdered preparation to completely dissolve into the liquid medium).

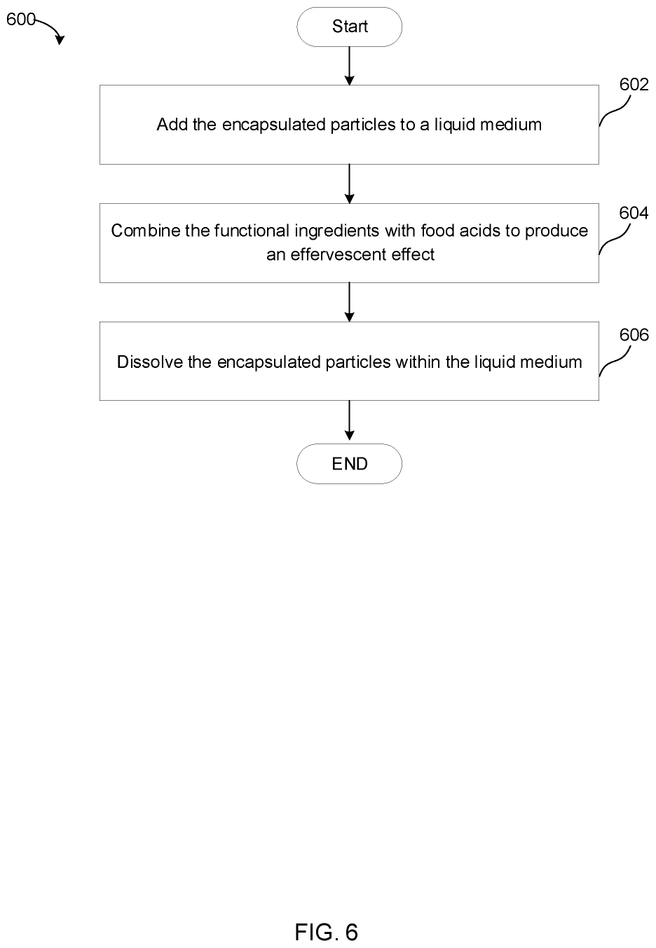

FIG. 6 illustrates a method 600 of producing a cannabinoid preparation according to an embodiment of the disclosure. In some cases, method 600 implements one or more aspects of the figures described herein, including FIGS. 3, 4, and 5.

In some cases, the method 600 may comprise adding 602 the encapsulated particles to a liquid medium. One non-limiting example of a liquid medium may include water (e.g., mineral water, tap water, carbonated or sparkling water, etc.).

Further, the method 600 may comprise combining 604 the functional ingredients, previously described in relation to FIG. 5, with food acids to produce an effervescent effect.

In some cases, the method may then comprise dissolving 606 the encapsulated particles within the liquid medium. As noted above, in some cases, the effervescent effect produced by combining 604 the functional ingredients with food acids may serve to reduce the dissolution time of the powdered cannabinoid preparation (i.e., as compared to a powdered preparation that does not exhibit an effervescent effect when added to a liquid medium).

### Additional Embodiments

In some embodiments, the preparation may further comprise an encapsulant; wherein, the encapsulant is coupled to an outer surface of one or more of the at least one of a cannabinoid or a cannabinoid extract containing one or more cannabinoids and the plurality of excipient particles. In some cases, the carbonate salt comprises potassium bicarbonate, calcium carbonate. In some examples, the targeted pH range when the dry consumable preparation is added to a targeted amount of water is between 2 and 6. In some examples, the ratio of the carbonate salt to the at least one acid comprises a first ratio, the first ratio comprising a dry weight ratio. In some cases, the preparation is configured to induce a pharmacokinetic profile when the preparation is ingested.

In some embodiments, the plurality of excipient particles comprise one of a carbohydrate sweetener, a water-soluble powdered flavor, and a water-soluble powdered color. In some embodiments, the encapsulant comprises a water-soluble starch. In some cases, the conventional pharmacokinetic profile comprises a bell-curve shape onset and decline, and a total time of four to six hours.

In some cases, the encapsulant comprises an oxidative barrier, a flow aid, a bulking agent, a dissolution agent, and a carrier of active ingredients, wherein, the active ingredients comprise one or more of dietary mineral content and an effervescence agent. In some cases, the carbohydrate sweetener comprises at least one of sucrose, fructose, glucose, corn syrup, corn syrup solids, lactose, honey, agave nectar, allulose, maltodextrin, tapioca syrup, digestion resistant maltodextrin, trehalose, galactose, and starch. In some cases, the onset comprises an effect realization with one hour. In some cases, the decline comprises an effect offset realization within five hours.

In some examples, the dry consumable preparation comprises 10% maltodextrin by weight. In some cases, the dry consumable preparation further comprises a nutraceutical composition, the nutraceutical composition having an amount of Quillaja selected to impart a stabilizing and emulsifying effect when the preparation is mixed with the targeted amount of water. One such amount preferably comprises a ratio range of 1:1 to 1:4 and more preferably comprises a ratio of 1:2 ratio of quillaja extract:oil to impart the stabilizing and emulsifying effect. However, the amount of quillaja will vary depending on the potency and oil quantity. One target amount of quillaja is 0.05%, with a range from 0.03% to 0.07% by dry weight In some cases, the dry consumable preparation further comprises one or more herbal extracts comprising at least one of cayenne extract, lemon oil, turmeric extract, cinnamon oil, bromelain, lavender oil, tart cherry, passion flower extract, and an essential oil. Additionally, or alternatively, the dry consumable preparation comprises one or more nutrient vitamins or minerals comprising at least one of L-Theanine, Vitamin C, Folic Acid, Vitamin D, Glucosamine Sulfate, Biotin, Vitamins A, B1, B2, B3, B6, B12, and zinc gluconate. In some embodiments, the at least one acid comprises tartaric acid, wherein a ratio of the tartaric acid to the preparation comprises a second ratio, wherein the second ratio imparts a desired taste effervescent quality and a desired period of time of effervescence when the preparation is mixed with the targeted amount of water.

In some embodiments, the powder composition is configured to form a stable, effervescent emulsion when mixed with the targeted amount of water. In one such embodiment, one “serving” of powder composition may comprise 7.5 g total powder in 4 to 6 oz of water. Therefore, the approximate ratio of water:powder would range between 10:1 to 30:1, more preferably between 15:1 to 25:1, and most preferably between 16:1 to 24:1.

In some embodiments, the plurality of excipient particles comprises an oil adsorbent agent and carbohydrate sweetener, wherein the carbohydrate sweetener comprises sucrose and fructose. In some cases, a ratio of the sucrose to the fructose is about 1:1. Alternatively, the ratio of the sucrose to fructose comprises a third ratio. In some cases, the preparation comprises at least 30% by weight fructose and at least 30% by weight fructose.

In some cases, the dry consumable preparation comprises one of between about 0.05% and about 0.73% by weight CBD oil, about 0.155% by weight CBD oil, and about 0.15% by weight CBD oil. In some embodiments, the plurality of excipient particles comprising an oil adsorbent agent comprises a first size. In some embodiments, plating the at least one of the cannabinoid or the cannabinoid extract containing one or more cannabinoids onto the plurality of excipient particles comprising an oil absorbent agent comprises expanding the plurality of excipient particles comprising an oil adsorbent agent to a second size, wherein the second size is less than 1.5 times the first size.

In some embodiments, the plurality of excipient particles comprising an oil adsorbent agent comprises one of a silica and a modified food starch, a surface, an interior, and a plurality of pores and capillaries extending from the surface to the interior. Further, the at least one of the cannabinoid or the cannabinoid extract containing one or more cannabinoids plated to the plurality of excipient particles comprises a first portion plated to the surface and a second portion plated to the plurality of pores and capillaries extending from the surface to the interior.

In some cases, the oil absorbent agent comprises one of maltodextrin, cyclodextrin, and a polysaccharide gum. In some cases, the polysaccharide gum comprises acacia gum. In some cases, the cannabinoid or the cannabinoid extract containing one or more cannabinoids comprises cannabinoid oil. Further, a ratio of at least a portion of the plurality of excipient particles to the at least one of a cannabinoid and a cannabinoid extract containing one or more cannabinoids ranges from 400:1 to 600:1, wherein the ratio of at least a portion of the plurality of excipient particles to the at least one of a cannabinoid and a cannabinoid extract containing one or more cannabinoids comprises a fourth ratio.

In some cases, the dry consumable preparation comprises one of a 3D printed food additive, a pressed tablet, and a powder adapted for receipt by a water dissolvable capsule.

In one example, a dry consumable preparation is disclosed. The exemplary preparation has a bulking agent. The exemplary preparation also has at least one of a cannabinoid or a cannabinoid extract containing one or more cannabinoids. The at least one of the cannabinoid or the cannabinoid extract containing one or more cannabinoids is plated onto the bulking agent. The exemplary preparation also has an effervescence agent, the effervescence agent having sodium bicarbonate, potassium bicarbonate, and at least one acid, the at least one acid having at least one of citric acid, tartaric acid, or malic acid, the effervescence agent further having a ratio of the sodium bicarbonate to the potassium bicarbonate to the at least one acid. The ratio is configured to create a chemical pH buffering system at a targeted pH range when the dry consumable preparation is added to a targeted amount of water.

In some cases, the method may further comprise adding the encapsulated particles to a liquid medium; combining the functional ingredients with food acids to produce an effervescent effect; and dissolving the encapsulated particles within the liquid medium.

In some cases, the method may further comprise retaining a greater than 85% of a stated cannabinoid potency for the cannabinoid preparation two years after producing the cannabinoid preparation.

An exemplary method of making a dry consumable preparation is also disclosed. The exemplary method includes mixing at least one of a cannabinoid or a cannabinoid extract containing one or more cannabinoids with a bulking agent, whereby the at least one of the cannabinoid or the cannabinoid extract containing one or more cannabinoids is plated onto the bulking agent. The exemplary method also includes introducing an effervescence agent to the mixture, the effervescence agent having sodium bicarbonate, potassium bicarbonate, and at least one acid. The at least one acid has at least one of citric acid, tartaric acid, or malic acid. The effervescence agent further has a ratio of the sodium bicarbonate to the potassium bicarbonate to the at least one acid that is configured to create a chemical pH buffering system at a targeted pH range when the dry consumable preparation is added to a targeted amount of water.

Those skilled in the art can readily recognize that numerous variations and substitutions may be made in the invention, its use, and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention as expressed in the claims.

What is claimed is:

1. A dry consumable preparation comprising:
a plurality of excipient particles;
at least one of a cannabinoid and a cannabinoid extract containing one or more cannabinoids, the at least one of the cannabinoid or the cannabinoid extract containing one or more cannabinoids plated to the plurality of excipient particles;
an effervescence agent, the effervescence agent comprising a carbonate salt and at least one acid; wherein,
the carbonate salt comprises at least one of sodium, potassium and calcium;
the at least one acid comprises at least one of citric acid, tartaric acid, lactic, acetic, benzoic, ascorbic, oxalic, tannic, phosphoric, or malic acid; and
a ratio of the carbonate salt to the at least one acid is at least 1:1, the ratio configured to create a chemical pH buffering system at a targeted pH range when the dry consumable preparation is added to a targeted amount of water.

2. The preparation of claim 1, further comprising,
an encapsulant; wherein,
the encapsulant is coupled to an outer surface of one or more of the at least one of a cannabinoid or a cannabinoid extract containing one or more cannabinoids and the plurality of excipient particles;
the carbonate comprises potassium bicarbonate and calcium carbonate salt;
the targeted pH range is between 2 and 6;
the ratio of the carbonate to the at least one acid comprises a first ratio, the first ratio comprising a dry weight ratio; and
the preparation is configured to induce a conventional bell pharmacokinetic profile when the preparation is ingested.

3. The preparation of claim 2 wherein,
the plurality of excipient particles comprise one of a carbohydrate sweetener, a water-soluble powdered flavor, and a water-soluble powdered color;
the encapsulant comprises a water-soluble starch; and
the conventional bell pharmacokinetic profile comprises a total time of four to six hours for an onset and decline.

4. The preparation of claim 3 wherein,
the encapsulant comprises an oxidative barrier, a flow aid, a bulking agent, a dissolution agent, and a carrier of active ingredients, wherein, the active ingredients comprise one or more of dietary mineral content and an effervescence agent;
the carbohydrate sweetener comprises at least one of sucrose, fructose, glucose, corn syrup, corn syrup solids, lactose, honey, agave nectar, allulose, maltodextrin, tapioca syrup, digestion resistant maltodextrin, trehalose, galactose, and starch;
the linear onset comprises an effect realization with one hour; and
the linear decline comprises an effect offset realization within five hours.

5. The preparation of claim 4 further comprising:
10% maltodextrin by weight;
a nutraceutical composition, the nutraceutical composition having an amount of Quillaja selected to impart a stabilizing and emulsifying effect when the preparation is mixed with the targeted amount of water;
one or more herbal extracts comprising at least one of cayenne extract, lemon oil, turmeric extract, cinnamon oil, bromelain, lavender oil, tart cherry, passion flower extract, and an essential oil;
one or more nutrient vitamins or minerals comprising at least one of L-Theanine, Vitamin C, Folic Acid, Vitamin D, Glucosamine Sulfate, Biotin, Vitamins A, B1, B2, B3, B6, B12, and zinc gluconate; wherein
the at least one acid comprises tartaric acid;
a ratio of the tartaric acid to the preparation comprises a second ratio, wherein the second ratio imparts a desired taste quality when the preparation is mixed with the targeted amount of water; and
the powder composition is configured to form a stable, effervescent emulsion when mixed with the targeted amount of water.

6. The preparation of claim 2 wherein,
the plurality of excipient particles comprises an oil adsorbent agent and carbohydrate sweetener;
the carbohydrate sweetener comprises sucrose and fructose;
a ratio of the sucrose to the fructose comprises about 1:1, the ratio of the sucrose to fructose comprising a third ratio;
the preparation comprises at least 30% by weight fructose and at least 30% by weight fructose; and
one of,
between about 0.05% and about 0.73% by weight CBD oil,
about 0.155% by weight CBD oil, and
about 0.15% by weight CBD oil.

7. The preparation of claim 6 wherein,
the plurality of excipient particles comprising an oil adsorbent agent comprises a first size;
plating the at least one of the cannabinoid or the cannabinoid extract containing one or more cannabinoids onto the plurality of excipient particles comprising an oil absorbent agent comprises expanding the plurality of excipient particles comprising an oil adsorbent agent to a second size; and
the second size is less than 1.5 times the first size.

8. The preparation of claim 7 wherein,
the plurality of excipient particles comprising an oil adsorbent agent comprises,
one of a silica and a modified food starch,
a surface,
an interior, and
a plurality of pores and capillaries extending from the surface to the interior; and
the at least one of the cannabinoid or the cannabinoid extract containing one or more cannabinoids plated to the plurality of excipient particles comprises,
a first portion plated to the surface, and
a second portion plated to the plurality of pores and capillaries extending from the surface to the interior.

9. The preparation of claim 8 wherein,
the oil absorbent agent comprises one of maltodextrin, cyclodextrin, and a polysaccharide gum;
the cannabinoid or the cannabinoid extract containing one or more cannabinoids comprises cannabinoid oil; and further comprising,
a ratio of at least a portion of the plurality of excipient particles to the at least one of a cannabinoid and a cannabinoid extract containing one or more cannabinoids ranges from 400:1 to 600:1, wherein the ratio of at least a portion of the plurality of excipient particles to the at least one of a cannabinoid and a cannabinoid extract containing one or more cannabinoids comprises a fourth ratio.

10. The preparation of claim 9 wherein the polysaccharide gum comprises acacia gum.

11. The preparation of claim 10, wherein the preparation comprises one of:
a 3D printed food additive;
a pressed tablet; and
a powder adapted for receipt by a water-dissolvable capsule.

12. A method of producing a cannabinoid preparation comprising,
providing to a fluid bed dryer a powdered preparation, wherein,
the powdered preparation comprises a plurality of excipient particles and at least one of a cannabinoid and a cannabinoid extract containing one or more cannabinoids plated to the plurality of excipient particles,
the plurality of excipient particles comprise,
an oil adsorbent agent, and
an outer surface having one or more pores and capillaries, and
the at least one of the cannabinoid or the cannabinoid extract containing one or more cannabinoids plated to the plurality of excipient particles comprises at least one of the cannabinoid or the cannabinoid extract containing one or more cannabinoids plated to one of the outer surface, pores, and capillaries;
spraying an aqueous solution onto the outer surface;
drying the particles; and
encapsulating the particles with a layer of functional ingredients.

13. The method of claim 12, further comprising,
adding the encapsulated particles to a liquid medium;
combining the functional ingredients with food acids to produce an effervescent effect; and
dissolving the encapsulated particles within the liquid medium.

14. The method of claim 12, further comprising: retaining a greater than 85% of a stated cannabinoid potency for the cannabinoid preparation two years after producing the cannabinoid preparation.
